(19) United States
(12) Patent Application Publication
(10) Pub. No.: US 2007/0225246 A1
(43) Pub. Date: Sep. 27, 2007

(54) O-acetyl-ADP-ribose non-hydrolyzable analogs

(76) Inventors: John M. Denu, McFarland, WI (US); Lindsay R. Comstock, Madison, WI (US)

Correspondence Address: BRINKS HOFER GILSON & LIONE, P.O. BOX 10395, CHICAGO, IL, 60610, US

(21) Appl. No.: 11/728,503

(22) Filed: Mar. 26, 2007

Related U.S. Application Data
(60) Provisional application No. 60/786,444, filed on Mar. 27, 2006.

(51) Int. Cl. A61K 31/7076; C07H 19/04

(52) U.S. Cl. 514/47; 536/26.7

(57) ABSTRACT

Compounds, compositions and methods for modulating cell death in target cells, particularly cancer cells are provided. The compounds are analogs of O-acetyl-ADP-ribose (OAADPr).

### CROSS-REFERENCE TO RELATED APPLICATIONS

This invention claims priority to U.S. Provisional Patent Application Ser. No. 60/786,444, filed Mar. 27, 2006.

### FEDERALLY SPONSORED RESEARCH OR DEVELOPMENT

This invention was made with United States government support from the National Institutes of Health (NIH), grant numbers GM065386 and GM059785. The United States government may have certain rights in this invention.

### FIELD OF INVENTION

This invention relates to the fields of biochemistry and oncology. More specifically, the present invention provides O-acetyl-ADP-ribose non-hydrolyzable analogs, compositions and methods for modulating cell death in target cells, particularly cancer cells.

### BACKGROUND

Several publications are referenced in this application by numbers in parentheses in order to more fully describe the state of the art to which this invention pertains. The disclosure of each of these publications is incorporated by reference herein.

Post-translational modifications found on histone protein play a critical role in transcriptional regulation. Mainly, the acetylation states of histones have been identified as a key contributor in governing transcription, DNA synthesis and repair. Dynamic acetylation is controlled by histone acetyltransferases (HATs) and histone deacetylases (HDACs). Although HATs and HDACs have been investigated extensively and are shown to play an important role in gene silencing, the evaluation of HDACs is of high interest due to their direct link to human disease. Protein deacetylases have been implicated in a variety of disease states including aging, diabetes, HIV regulation, cancer, cardiovascular disorders, and neurodegenerative diseases. Histone deacetylase inhibitors are currently in clinical trials as cancer treatments.

To date, three classes of HDACs have been shown to play a critical role in the regulation of transcription. Class I and II share a conserved catalytic domain and function via a zinc-mediated hydrolysis reaction to afford the deacetylated substrate and acetate. Class III HDACs, the sirtuin family of histone/protein deacetylases, deacetylate via an alternate mechanism.

Silent information regulator 2 (Sir2) proteins (also referred to as “sirtuins”) are well conserved across all kingdoms of life and are implicated in the control of gene silencing, apoptosis, metabolism, and aging (Moazed, 2001, Curr. Opin. Cell Biol. 13: 232-238); Gasser and Cockell, 2001, Gene 279: 1-16); Brunet et al., 2004, Science 303: 2011-2015; Motta et al., 2004, Cell 116: 551-563; Starai et al., 2004, Curr. Opin. Microbiol. 7: 115-119; Hekimi and Guarente, 2003, Science 299: 1351-1354). Life-span increases in yeast, flies and worms caused by caloric restriction or by natural antioxidants (e.g. resveratrol), require Sir2 (Howitz et al., 2003, Nature 425: 191-196; Wood et al., 2004, Nature 430: 686-689). Among the seven mammalian Sir2 homologs, SIRT1/Sir2α regulates skeletal muscle differentiation (Fulco et al., 2003, Mol. Cell. 12: 51-62), represses damage-responsive Forkhead transcription factors (Brunet et al., 2004, Science 303: 2011-2015; Motta et al., 2004, Cell 116: 551-563), negatively controls p53 to promote cell survival under stress (reviewed in Smith, 2002, Trends Cell Biol. 12: 404-406), and promotes fat mobilization in white adipocytes (Picard et al., 2004, Nature 429: 771-776). Human SIRT2 is associated with microtubules in the cytoplasm and can deacetylate α-tubulin (North et al., 2003, Mol. Cell. 11: 437-444). Though the role of mitochrondrial SIRT3 is unknown (Onyango et al., 2002, Proc. Natl. Acad. Sci. USA 99: 13653-13658; Schwer et al., 2002, J. Cell Biol 158: 647-657), variability of the human SIRT3 gene is associated with survivorship in the elderly (Rose et al., 2003, Exp. Gerontol 38: 1065-1070).

Sirtuins catalyze a unique protein deacetylation reaction that requires the co-enzyme NAD⁺, a key intermediate in energy metabolism. In this reaction, nicotinamide (vitamin B3) is liberated from NAD⁺ and the acetyl-group of substrate is transferred to cleaved NAD⁺, generating a novel metabolite O-acetyl-ADP ribose, OAADPr (Tanner et al., 2000, Proc. Natl. Acad. Sci. USA 97:14178-14182; Tanny and Moazed, 2001, Proc. Natl. Acad. Sci. USA 98: 415-420; Sauve et al., 2001, Biochemistry 40: 15456-15463; Jackson and Denu, 2002, J. Biol. Chemistry 277: 18535-18544; Denu, 2003, TIBS 28: 41-48). Surprisingly, although genetic studies have linked sirtuins to diverse phenotypes, few reports have investigated the biological function(s) of OAADPr and its possible connection with the observed sirtuin-dependent biology. It has been suggested that OAADPr might be a substrate for other linked enzymatic processes, an allosteric regulator, or a second messenger. The first report of bio-activity came from the observation that OAADPr injected into oocytes or blastomeres caused a block/delay in maturation and cell division, respectively (Borra et al., 2002, J. Biol. Chem. 277: 12632-12641). Enzymes capable of metabolizing OAADPr have been detected in several diverse cells (Rafty et al., 2002, Journal of Biological Chemistry 277: 47114-47122). In vitro, select members of the ADP-ribose hydrolase (Nudix) family of enzymes (e.g., mNudT5 and yeast YSA1) are capable of efficient hydrolysis of OAADPr, while others like human Nudt9 are not (Rafty et al., 2002, Journal of Biological Chemistry 277: 47114-47122).

Sirtuins are efficient NAD⁺-dependent deacetylases and the reaction is coupled with the formation of OAADPr as the primary product. The unique cellular function of OAADPr may be linked to the observed physiological effects of the sirtuins, including gene silencing. Although it was initially believed that OAADPr may be a substrate for other linked enzymatic processes or serve as an allosteric regulator or second messenger, efforts thus for to elucidate its role in cellular function has been restricted by both the instability and quantity of native OAADPr.

### BRIEF SUMMARY

The present invention is directed to compounds and pharmaceutically acceptable salts thereof, compositions, and methods useful in modulating cell death in target cells, particularly cancer cells, and should have therapeutic value in treating disorders associated with aberrant cell proliferation, such as cancer. The compounds of the present invention should also have therapeutic values in treating disorders such as diabetes.

In one embodiment, the present invention provides compounds represented by formula (I) or salts thereof:

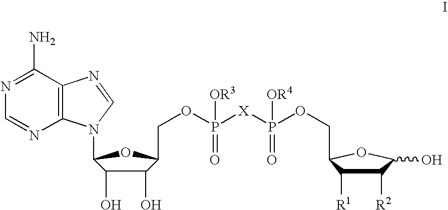

where X is O or CH₂; and

R¹ and R² are each independently selected from the group consisting of hydrogen, —F, —OH, —OR^{a}, —SR^{a}, —NH₂, —NHC(O)CH₃, —CH₂C(O)CH₃ and —CH₂C(CH₃)═CH₂;

where R^{a} is selected from the group consisting of substituted or unsubstituted C₁₋₈ alkyl, substituted or unsubstituted C₂₋₈ alkenyl, substituted or unsubstituted C₂₋₈ alkynyl; and

R³ and R⁴ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;

with the proviso that at least one of R¹ and R² is other than hydrogen, —F, —OH, or —NH₂; and

with the proviso that excuded from the scope of formula I is O-acetyl ADP ribose.

In another aspect, the present invention provides compositions useful in modulating cell death in target cells, particularly cancer cells. The present invention also provides compositions useful for the treatment of diabetes. In one embodiment, a composition according to the present invention comprises a compound according to the invention and a pharmaceutically acceptable carrier or excipient.

In another aspect, the present invention provides a method for modulating cell death in a target cell. The method comprises contacting a cell expressing O-acetyl-ADP-ribose with a compound of formula (I), or a salt thereof.

In another aspect, the present invention provides a method for treating an O-acetyl-ADP-ribose-mediated condition. The method comprises administering to a subject an effective amount of a compound of formula (I), or a salt thereof.

In another aspect, the present invention provides a method for producing 2-N-acetyl-2-deoxy-D-ribofuranose 5-hydrogen phosphate. The method comprises providing 1,2:5,6-di-O-isopropylidene-3-O-triflate-α-D-glucofuranose; transforming 1,2:5,6-di-O-isopropylidene-3-O-triflate-α-D-glucofuranose to 2-N-acetyl-2-deoxy-D-ribofuranose 5-hydrogen phosphate; and isolating 2-N-acetyl-2-deoxy-D-ribofuranose 5-hydrogen phosphate.

In another aspect, the present invention provides a method for producing 3-N-acetyl-3-deoxy-D-ribofuranose 5-hydrogen phosphate. The method comprises providing 5-O-benzyl-3-triflyl-1,2-O-isopropylidene-α-D-xylofuranose; transforming 5-O-benzyl-3-triflyl-1,2-O-isopropylidene-α-D-xylofuranose to 3-N-Acetyl-3-deoxy-D-ribofuranose 5-hydrogen phosphate; and isolating 3-N-Acetyl-3-deoxy-D-ribofuranose 5-hydrogen phosphate.

### DETAILED DESCRIPTION

Definitions

When describing the compounds, compositions, methods and processes of this invention, the following terms have the following meanings, unless otherwise indicated.

“OAADPr” as used herein refers to O-acetyl-ADP-ribose and includes 2′-O-acetyl-ADP-ribose and 3′-O-acetyl-ADP-ribose, which may be referred to as 2′-OAADPr and 3′-OAADPr, respectively.

“2′-NAADPr” as used herein refers to 2′-N-acetyl-ADP-ribose.

“3′-NAADPr” as used herein refers to 3′-N-acetyl-ADP-ribose.

“ADPr” as used herein refers to ADP-ribose.

“Alkyl” by itself or as part of another substituent refers to a hydrocarbon group which may be linear, cyclic, or branched or a combination thereof having the number of carbon atoms designated (i.e., C₁₋₈ means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc. Alkyl groups can be substituted or unsubstituted, unless otherwise indicated. Examples of substituted alkyl include haloalkyl, thioalkyl, aminoalkyl, and the like.

“Alkenyl” refers to an unsaturated hydrocarbon group which may be linear, cyclic or branched or a combination thereof. Alkenyl groups with 2-8 carbon atoms are preferred. The alkenyl group may contain 1, 2 or 3 carbon-carbon double bonds. Examples of alkenyl groups include ethenyl, n-propenyl, isopropenyl, n-but-2-enyl, n-hex-3-enyl, cyclohexenyl, cyclopentenyl and the like. Alkenyl groups can be substituted or unsubstituted, unless otherwise indicated.

“Alkynyl” refers to an unsaturated hydrocarbon group which may be linear, cyclic or branched or a combination thereof. Alkynyl groups with 2-8 carbon atoms are preferred. The alkynyl group may contain 1, 2 or 3 carbon-carbon triple bonds. Examples of alkynyl groups include ethynyl, n-propynyl, n-but-2-ynyl, n-hex-3-ynyl and the like. Alkynyl groups can be substituted or unsubstituted, unless otherwise indicated.

“Aryl” refers to a polyunsaturated, aromatic hydrocarbon group having a single ring (monocyclic) or multiple rings (bicyclic), which can be fused together or linked covalently. Aryl groups with 6-10 carbon atoms are preferred, where this number of carbon atoms can be designated by C₆₋₁₀, for example. Examples of aryl groups include phenyl and naphthalene-1-yl, naphthalene-2-yl, biphenyl and the like. Aryl groups can be substituted or unsubstituted, unless otherwise indicated.

“Heterocyclyl” refers to a saturated or unsaturated non-aromatic ring containing at least one heteroatom (typically 1 to 5 heteroatoms) selected from nitrogen, oxygen or sulfur. The heterocyclyl ring may be monocyclic or bicyclic. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Examples of heterocycle groups include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene, quinuclidine and the like. Preferred heterocyclic groups are monocyclic, though they may be fused or linked covalently to an aryl or heteroaryl ring system.

“Heteroaryl” refers to an aromatic group containing at least one heteroatom, where the heteroaryl group may be monocyclic or bicyclic. Examples include pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl.

Heterocyclyl and heteroaryl can be attached at any available ring carbon or heteroatom. Each heterocyclyl and heteroaryl may have one or more rings. When multiple rings are present, they can be fused together or linked covalently. Each heterocyclyl and heteroaryl must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Heterocyclyl and heteroaryl groups can be substituted or unsubstituted, unless otherwise indicated. For substituted groups, the substitution may be on a carbon or heteroatom. For example, when the substitution is oxo (═O or —O⁻), the resulting group may have either a carbonyl (—C(O)—) or a N-oxide (—N⁺—O⁻).

Suitable substituents for substituted alkyl, substituted alkenyl, and substituted alkynyl include halogen, —CN, —CO₂R′, —C(O)R′, —C(O)NR′R″, oxo (═O or —O⁻), —OR′, —OC(O)R′, —OC(O)NR′R″, —NO₂, —NR′C(O)R″, —NR′″C(O)NR′R″, —NR′R″, —NR′CO₂R″, —NR′S(O)R″, —NR′S(O)₂R′″, —NR′″S(O)NR′R″, —NR′″S(O)₂NR′R″, —SR′, —S(O)R′, —S(O)₂R′, —S(O)₂NR′R″, —NR′—C(NHR″)═NR′″, —SiR′R″R′″, —N₃, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to (2m′+1), where m′ is the total number of carbon atoms in such radical.

Suitable substituents for substituted aryl, substituted heteroaryl and substituted heterocyclyl include halogen, —CN, —CO₂R′, —C(O)R′, —C(O)NR′R″, oxo (═O or —O⁻), —OR′, —OC(O)R′, —OC(O)NR′R″, —NO₂, —NR′C(O)R″, —NR′″C(O)NR′R″, —NR′R″, —NR′CO₂R″, —NR′S(O)R″, —NR′S(O)₂R″, —NR′″S(O)NR′R″, —NR′″S(O)₂NR′R″, —SR′, —S(O)R′, —S(O)₂R′, —S(O)₂NR′R″, —NR′—C(NHR″)═NR′″, —SiR′R″R′″, —N₃, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10 membered heterocyclyl. The number of possible substituents range from zero to the total number of open valences on the aromatic ring system.

As used above, R′, R″ and R′″ each independently refer to a variety of groups including hydrogen, substituted or unsubstituted C₁₋₈ alkyl, substituted or unsubstituted C₂₋₈ alkenyl, substituted or unsubstituted C₂₋₈ alkynyl, substituted or unsubstituted aryl, substituted or unsubstituted heteroaryl, substituted or unsubstituted heterocyclyl, substituted or unsubstituted arylalkyl, substituted or unsubstituted aryloxyalkyl. When R′ and R″ are attached to the same nitrogen atom, they can be combined with the nitrogen atom to form a 3-, 4-, 5-, 6-, or 7-membered ring (for example, —NR′R″ includes 1-pyrrolidinyl and 4-morpholinyl). Furthermore, R′ and R″, R″ and R′″, or R′ and R′″ may together with the atom(s) to which they are attached, form a substituted or unsubstituted 5-6- or 7-membered ring.

Two of the substituents on adjacent atoms of an aryl or heteroaryl ring may optionally be replaced with a substituent of the formula -T-C(O)—(CH₂)_{q}—U—, wherein T and U are independently —NR″″, —O—, —CH₂— or a single bond, and q is an integer of from 0 to 2. Alternatively, two of the substituents on adjacent atoms of the aryl or heteroaryl ring may optionally be replaced with a substituent of the formula -A′-(CH₂)ᵣ—B′—, wherein A′ and B′ are independently —CH₂—, —O—, —NR″″—, —S—, —S(O)—, —S(O)₂—, —S(O)₂NR″″— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR″″—, —S—, —S(O)—, —S(O)₂—, or —S(O)₂NR′—. R″″ in is selected from hydrogen or unsubstituted C₁₋₈ alkyl.

“Cancer” and “cancerous” refer to or describe the physiological condition in mammals that is typically characterized by unregulated cell growth. The cells that divide and grow uncontrollably invade and disrupt other tissues and spread to other areas of the body (metastasis) through the lymphatic system or the blood stream. Examples of cancer include but are not limited to, carcinoma, lymphoma, blastoma, sarcoma, and leukemia. More particular examples of such cancers include prostate cancer, colon cancer, squamous cell cancer, small-cell lunge cancer, non-small cell lunar cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma kidney cancer, liver cancer, vulval cancer, thyroid cancer, hepatic carcinoma and various types of head and neck cancer. Cancer exerts its deleterious effect on the body by 1) destroying the surrounding adjacent tissues: e.g. compressing nerves, eroding blood vessels, or causing perforation of organs; and 2) replacing normal functioning cells in distant sites: e.g. replacing blood forming cells in the bone marrow, replacing bones leading to increased calcium levels in the blood, or in the heart muscles so that the heart fails.

The term “induces cell death” or “capable of inducing cell death” refers to the ability of a compound of the present invention to make a viable cell become nonviable.

The phrase “induces apoptosis” or “capable of inducing apoptosis” refers to the ability of a compound of the present invention to induce programmed cell death.

“Diabetes” (or “diabetes mellitus”) is a metabolic disorder characterized by hyperglycemia (high blood sugar) and other signs. “Insulin” is a polypeptide that regulates carbohydrate metabolism. Insulin is used to treat some forms of diabetes.

“Pharmaceutically acceptable” carrier, diluent, or excipient is a carrier, diluent, or excipient compatible with the other ingredients of the formulation and not deleterious to the recipient thereof.

“Pharmaceutically-acceptable salt” refers to a salt which is acceptable for administration to a patient, such as a mammal (e.g., salts having acceptable mammalian safety for a given dosage regime). Such salts can be derived from pharmaceutically-acceptable inorganic or organic bases and from pharmaceutically-acceptable inorganic or organic acids, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine., methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Salts derived from pharmaceutically-acceptable acids include acetic, ascorbic, benzenesulfonic, benzoic, camphosulfonic, citric, ethanesulfonic, fumaric, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, tartaric, p-toluenesulfonic and the like.

Also included are salts of amino acids such as arginate and the like, and salts of organic acids like glucuronic or galactunoric acids and the like (see, for example, Berge et al., 1977, J. Pharmaceutical Science 66: 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Salt thereof” refers to a compound formed when the hydrogen of an acid is replaced by a cation, such as a metal cation or an organic cation and the like. Preferably, the salt is a pharmaceutically-acceptable salt, although this is not required for salts of intermediate compounds which are not intended for administration to a patient.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

“Therapeutically effective amount” refers to an amount sufficient to effect treatment when administered to a patient in need of treatment. “Treating” or “treatment” as used herein refers to the treating or treatment of a disease or medical condition (such as aberrant cell proliferation, uncontrolled division of cells, and cancer) in a patient, such as a mammal (particularly a human or a companion animal) which includes: (a) ameliorating the disease or medical condition, i.e., eliminating or causing regression of the disease or medical condition in a patient; (b) suppressing the disease or medical condition, i.e., slowing or arresting the development of the disease or medical condition in a patient; or (c) alleviating the symptoms of the disease or medical condition in a patient.

“Tumor”, as used herein, refers to all neoplastic cell growth and proliferation, whether malignant or benign, and all pre-cancerous and cancerous cells and tissues

Certain compounds of the present invention can exist in unsolvated forms as well as solvated forms, including hydrated forms. In general, both solvated forms and unsolvated forms are intended to be encompassed within the scope of the present invention. Certain compounds of the present invention may exist in multiple crystalline or amorphous forms (i.e., as polymorphs). In general, all physical forms are equivalent for the uses contemplated by the present invention and are intended to be within the scope of the present invention.

It will be apparent to one skilled in the art that certain compounds of the present invention may exist in tautomeric forms, all such tautomeric forms of the compounds being within the scope of the invention. Certain compounds of the present invention possess asymmetric carbon atoms (optical centers) or double bonds; the racemates, diastereomers, geometric isomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Sirtuins histone deacetylases (HDACs) deacetylate acetylated peptides. Class III HDACs are unique because the deacetylation process is NAD⁺-dependent as shown in Scheme 1. Silent information regulator 2 (Sir2) proteins are histone/protein deacetylases that regulate gene silencing, apoptosis, metabolism, and aging. Sir2 (or sirtuins) proteins catalyze a unique protein deacetylation reaction that absolutely requires the co-enzyme NAD⁺ and generates a novel metabolite O-acetyl-ADP ribose, OAADPr.

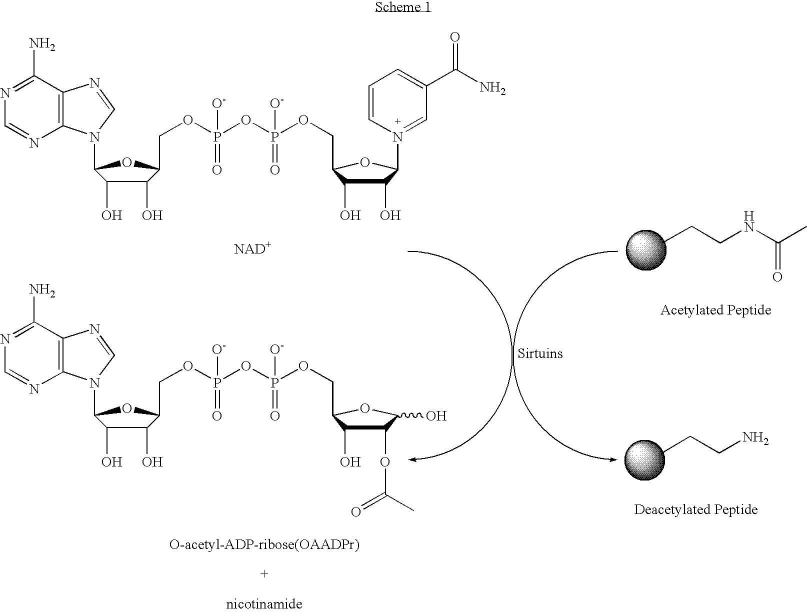

It is known that OAADPr directly activates the long transient receptor potential channel 2 (TRPM2), a Ca²⁺ permeable nonselective ion channel. TRPM2 channel over-stimulation by inhibiting the cellular breakdown of OAADPr leads to cell death, which is attenuated by a loss in endogenous levels of Sir2 homologues, SIRT2 and SIRT3. This data provides evidence for the role of OAADPr in controlling the TRPM2 channel, whose activity is known to confer susceptibility to cell death by oxidative stress and diabetogenic agents.

It is known that hydrolysis of both the bisphosphonate linkage and the critical acetyl functionality of OAADPr occurs by Nudix hydrolases and cellular esterases. Thus, to efficiently evaluate the role of OAADPr, non-hydrolyzable analogs must be synthesized which are not susceptible to Nudix hydrolases and cellular esterases. In addition, OAADPr is known to exist in ˜50:50 equilibrium between 2′-OAADPr and 3′-OAADPr (Sauve et al., 2001, Biochemistry 40: 15456-15463; Jackson and Denu, 2002, J. Biol. Chemistry 277: 18535-18544), so analogs of both forms are of interest in the present invention.

Compounds

The present invention provides compounds that are non-hydrolyzable analogs of O-acetyl-ADP-ribose (OAADPr). In one embodiment, the compounds of the present invention are represented by formula (I), or salts thereof:

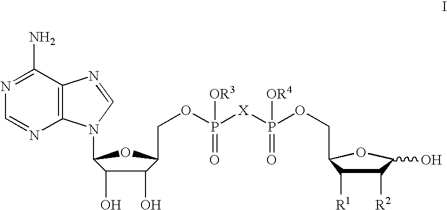

where X is O or CH₂; R¹ and R² are each independently selected from the group consisting of hydrogen, —F, —OH, —OR^{a}, —SR^{a}, —NH₂, —NHC(O)CH₃, —CH₂C(O)CH₃ and —CH₂C(CH₃)═CH₂; where R^{a} is selected from the group consisting of substituted or unsubstituted C₁₋₈ alkyl, substituted or unsubstituted C₂₋₈ alkenyl, substituted or unsubstituted C₂₋₈ alkynyl; and R³ and R⁴ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl; with the proviso that at least one of R¹ and R² is other than hydrogen, —F, —OH, or —NH₂; and with the proviso that excluded from the scope of formula I is O-acetyl ADP ribose.

In another embodiment, the compounds of the present invention are represented by formula (XI), or salts thereof:

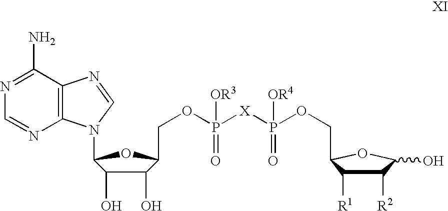

where X is O or CH₂; R¹ and R² are each independently selected from the group consisting of hydrogen, —F, —OH, —OR^{a}, —SR^{a}, —NH₂, —NHC(O)CH₃, —CH₂C(O)CH₃ and —CH₂C(CH₃)═CH₂; where R^{a} is selected from the group consisting of unsubstituted C₁₋₈ alkyl, substituted or unsubstituted C₂₋₈alkenyl, substituted or unsubstituted C₂₋₈alkynyl; and R³ and R⁴ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;

with the proviso that at least one of R¹ and R² is other than hydrogen, —F, —OH, or —NH₂.

In one embodiment of formulae (I and XI), at least one of R¹ and R² is other than hydrogen, —F, —OH, and —NH₂.

In one embodiment of formulae (I and XI), one of R¹ and R² is —OH and the other is other than hydrogen, —F, —OH, and —NH₂.

In another embodiment of formulae (I and XI), R¹ is OH, and R² is —NHC(O)CH₃.

In another embodiment of formulae (I and XI), R² is OH, and R¹ is —NHC(O)CH₃.

In another embodiment, the compound is of the formula (II):

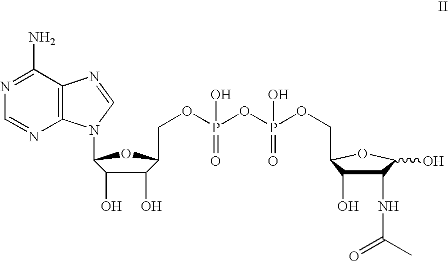

In another embodiment, the compound is of the formula (III):

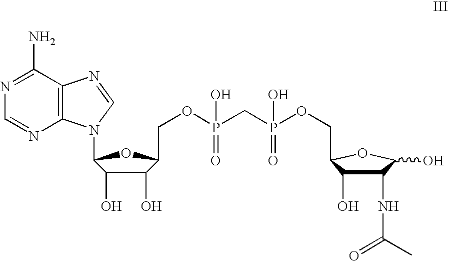

In another embodiment, the compound is of the formula (IV):

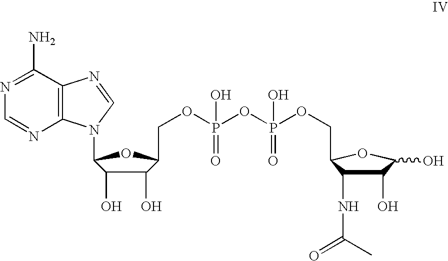

In another embodiment, the compound is of the formula (V):

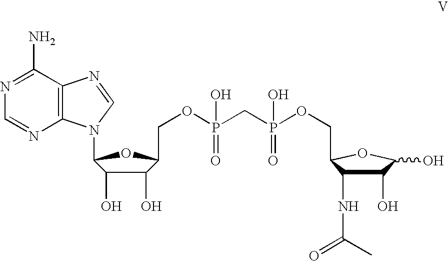

In one embodiment of formulae (I and XI), R³ and R⁴ are each independently selected from the group consisting of hydrogen, substituted or unsubstituted C₁₋₈ alkyl.

In one embodiment of formulae (I and XI), R³ and R⁴ are each independently selected from the group consisting of hydrogen, substituted or unsubstituted C₁₋₄ alkyl.

In one embodiment of formulae (I and XI), R³ and R⁴ are each hydrogen.

In one embodiment of formulae (I and XI), R³ and R⁴ are each independently C₁₋₈ alkyl.

In one embodiment of formulae (I and XI), R³ and R⁴ are each independently C₁₋₄ alkyl.

In one embodiment of formulae (I and XI), one of R³ and R⁴ is hydrogen, and the other is C₁₋₈ alkyl.

In one embodiment, the compound of formula (I-V or XI) is a salt. Preferably the salt is a base addition salt.

In another embodiment, the salt is derived from a pharmaceutically acceptable inorganic base.

In another embodiment, the salt is derived from a pharmaceutically acceptable inorganic base.

Compositions

In another aspect, the present invention provides compositions that modulate cell death. Generally, the compositions for modulating cell death activity in humans and animals will comprise a pharmaceutically acceptable excipient or diluent and a compound having the formula provided above as formula (I).

Yet in another aspect, the present invention provides compositions that can be used for the treatment of diabetes. Generally, the compositions for modulating cell death activity in humans and animals will comprise a pharmaceutically acceptable excipient or diluent and a compound having the formula provided above as formula (I).

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self emulsifications as described in U.S. Pat. No. 6,451,339, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated enterically or otherwise by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil in water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. Oral solutions can be prepared in combination with, for example, cyclodextrin, PEG and surfactants.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non toxic parenterally acceptable diluent or solvent, for example as a solution in 1,3-butane diol. Among the acceptable vehicles and solvents that may be employed are water, Ringer's solution and isotonic sodium chloride solution. In addition, sterile, axed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. Additionally, the compounds can be administered via ocular delivery by means of solutions or ointments. Still further, transdermal delivery of the subject compounds can be accomplished by means of iontophoretic patches and the like.

For topical use, creams, ointments, jellies, solutions or suspensions containing the compounds of the present invention are employed. As used herein, topical application is also meant to include the use of mouth washes and gargles.

The pharmaceutical compositions and methods of the present invention may further comprise other therapeutically active compounds as noted herein, such as those applied in the treatment of the above mentioned pathological conditions.

In yet another aspect, the present invention provides method of treating or preventing a condition by administering to a subject having such a condition a therapeutically effective amount of any compound of formula (I) above. Compounds for use in the present methods include those compounds according to formula (I), those provided above as embodiments, those specifically exemplified in the Examples below, and those provided with specific structures herein. The “subject” is defined herein to include animals such as mammals, including, but not limited to, primates (e.g., humans), cows, sheep, goats, horses, dogs, cats, rabbits, rats, mice and the like. In preferred embodiments, the subject is a human.

Depending on the disease to be treated and the subject's condition, the compounds and compositions of the present invention may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration and may be formulated, alone or together, in suitable dosage unit formulations containing conventional non toxic pharmaceutically acceptable carriers, adjuvants and vehicles appropriate for each rouse of administration. The present invention also contemplates administration of the compounds and compositions of the present invention in a depot formulation.

It will be understood that the specific dose level and frequency of dosage for any particular patient may be varied and will depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In one embodiment, the present invention provides a composition consisting of a pharmaceutically acceptable carrier and a compound of the invention.

Methods of Treatment

The present invention can be widely utilized for the treatment of various animal or human cancers or malignancies, including cancers associated with hematopoietic cells, central nervous system cells, lung cells, breast cells, ovary cells and liver cells. Specific human cancers amenable to treatment include melanoma, colon cancer, ovarian cancer, pancreatic cancer, stomach cancer, neuroblastoma, squamous cell carcinoma, fibrosarcoma and leukemia.

It can be determined if a compound of the present invention has activity by testing in a variety of methods against cancer. For example, the compound can be tested for activity by using the channel gating technique described in the experimental section.

It is also contemplated that the compounds of the present invention can be utilized for the treatment of diabetes. Nonselective cation channels, like TRPM2, are implicated in insulin secretion by regulating pancreatic beta-cell plasma membrane potential, Ca²⁺ homeostasis, and thus glucose signaling and homeostasis (Qian et al., 2002, Diabetes 51 Suppl 1:S183-189). TRPM2 is expressed in human islets, and is activated by OAADPr in an insulinoma cell line (Grubisha et al., 2006, J. Biol. Chem. 281: 14057-14065). TRPC-like channels and their activation by OAADPr-producing sirtuin enzymes may provide a mechanism for depolarization or Ca²⁺ entry in beta-cells (insulin producing cells), which could lead to the control of insulin secretion and overall glucose homeostasis. Consistent with this idea, overexpression of the sirtuin SIRT1 (BESTO mice) in beta-cells resulted in increased insulin secretion in response to glucose (Moynihanet et al., 2005, Cell Metab 2: 105-117). In another study, SIRT1^{-/-} mice and their isolated islets showed decreased insulin secretion (Bordone et al., 2006, PLoS Biol 4, e31). The sirtuin product OAADPr mediates, at least in part, these phenotypes brought about by modulating the levels of SIRT1 enzymes. Accordingly, OAADPr analogs of the present invention can act as agonists to stimulate insulin secretion in islets, by direct activation of the TRMP2 channel. Additionally, these OAADPr analogs can inhibit cellular enzymes that break down endogenous OAADPr, and thereby stimulate insulin secretion in islets. Thus, OAADPr analogs of the present invention can be used as insulin mimetics.

In one embodiment, the present invention provides a method of treating an O-acetyl-ADP-ribose-mediated condition involving administering to a subject a safe and effective amount of the compound or composition of the invention.

In one embodiment, the present invention provides a method of treating an O-acetyl-ADP-ribose-mediated condition involving administering to a subject a safe and effective amount of the compound or composition of the invention, where the O-acetyl-ADP-ribose-mediated condition is aberrant cell proliferation. Preferably the aberrant cell proliferation is cancer.

In one embodiment, the present invention provides a method of treating an O-acetyl-ADP-ribose-mediated condition involving administering to a subject a safe and effective amount of the compound or composition of the invention, where the O-acetyl-ADP-ribose-mediated condition is selected from the group consisting of aging, diabetes, HIV regulation, cancer, cardiovascular disorders, and neurodegenerative diseases.

In one embodiment, the present invention provides a method of modulating O-acetyl-ADP-ribose function in a cell, where the O-acetyl-ADP-ribose function in the cell is modulated by contacting the cell with a O-acetyl-ADP-ribose modulating amount of the compound of the present invention.

In one embodiment, the present invention provides a method for modulating cell death in a target cell, where the cell death is modulated by contacting a cell expressing O-acetyl-ADP-ribose with a compound of the present invention, thereby inducing cell death in said target cell.

In another embodiment, the present invention provides a method of inhibiting a tumor growth in a subject. The method comprises administering to the subject a pharmaceutical composition comprising a compound of the present invention in an amount sufficient to cause a reduction in the number of tumor cells, thereby inhibiting the tumor growth.

In another embodiment, the present invention provides a method of treating a patient having neoplasia. The method comprises administering to the patient in need thereof a pharmaceutical composition comprising a therapeutically effective amount of a compound of the present invention in a therapeutically effective amount sufficient to cause a reduction of the neoplastic cells in the patient.

In another embodiment, the present invention provides a method of inducing apoptosis in neoplastic cells in a subject. The method comprises administering to the subject a composition comprising a compound of the present invention in a dose effective to induce apoptosis in the neoplastic cells.

In another embodiment, the present invention provides a method of inducing apoptosis in tumor cells in a subject. The method comprises administering to the subject a composition comprising an effective amount of a compound of the present invention, thereby inducing apoptosis in the tumor cells. The method further comprises evaluating the cells for indication of apoptosis.

In another embodiment, the present invention provides a method of inhibiting a tumor growth in a subject. The method comprises administering to the subject a compound of the present invention in the amount sufficient to cause a reduction in the number of cells of the tumor, and thereby inhibiting the tumor growth.

In another embodiment, the present invention provides a method of treating a patient having neoplasia. The method includes administering to the patient in need thereof a pharmaceutical composition comprising a compound of the present invention in an amount which is effective to cause a reduction in the number of neoplastic cells in the patient.

In another embodiment, the present invention provides a method of inhibiting growth or proliferation of, or inducing reduction in the number of tumor cells in a subject, comprising administering to the subject a compound of the present invention, in an amount which is effective to inhibit growth or proliferation of the tumor cells.

In another embodiment, the present invention provides a method of inhibiting unwanted growth or proliferation of, or reducing the number of tumor cells in a human subject. The method includes administering to the human subject a compound of the present invention which is effective to cause inhibit the growth or proliferation of the established tumor, induce cell death of the established tumors, or to reduce the size of the established tumors.

Preparation of Compounds

The following examples are offered to illustrate, but not to limit, the claimed invention.

Additionally, those skilled in the art will recognize that the molecules claimed in this patent may be synthesized using a variety of standard organic chemistry transformations. Compounds of the invention can be made by the methods and approaches described in the following experimental section, and by the use of standard organic chemistry transformations that are well known to those skilled in the art.

Experimental

Reagents and solvents used below can be obtained from commercial sources such as Aldrich Chemical Co. (Milwaukee, Wis., USA) and used as received unless otherwise noted. The silica gel used in column flash chromatography was Merck no. 9385, 60 Å, 230-400 mesh. Reversed-phase flash silica was prepared and used as described in Kühler and Lindsten, 1983, Org. Chem. 48: 3589-3591; O'Neil, 1991, Synlett 9: 661-662. Analytical TLC was conducted on EM Science silica gel plates with detection by phosphomolybdic acid and/or UV light.

¹H-NMR and ¹³C-NMR spectra were recorded on a Varian MercuryPlus 300, Bruker AC300, or Varian Unity 500 spectrometer using TMS or solvent as the internal reference. Chemical shifts are reported in ppm, in δ units. Significant peaks are tabulated in the order: multiplicity (br, broad; s, singlet; d, doublet; t, triplet; q, quartet; m, multiplet) and number of protons. Mass spectra were obtained from the University of Wisconsin-Madison, Department of Chemistry or Biotechnology Center mass spectrometry facility. Mass spectrometry results are reported as the ratio of mass over charge, followed by the relative abundance of each ion (in parenthesis). A single m/e value is reported for the M+H (or, as noted, M−H, or M+Na) ion containing the most common atomic isotopes. Isotope patterns correspond to the expected formula in all cases.

Analytical HPLC was performed using a Shimadzu series 2010C HPLC with either a PolyHydroxyethyl A (HILIC) column (300 Å, 5 μm, 4.5×200 mm, PolyLC, Inc.) or C18 column (90 Å, 10 μm, 4.6×250 mm, Vydac) and detected at both 214 and 260 nm. All mobile phases were filtered through a Millipore 0.20-μm nylon filter prior to use. Compound separation for HILIC utilized a gradient system comprising of ACN (solvent A) and 10 mM NH₄OAc (solvent B) using a flow rate of 0.5 mL/min. The gradient was run isocratically in 20% B for 9 min followed by a linear gradient of 20-60% B over a 30-min period. Under these conditions, diadenosine 5′-pyrophosphate (AppA) and the desired acetylated ADPr co-eluted (23.5 min). Compound separation for C18 utilized a gradient system comprising of H₂O with 0.05% TFA (solvent A) and ACN with 0.02% TFA (solvent B) using a flow rate of 0.5 mL/min. The gradient was run isocratically in 0% B for 2 min followed by a linear gradient of 0-8% B over a 20-min period. The gradient was then increased to 100% over the next 5 min. Under these conditions, diadenosine 5′-pyrophosphate (AppA) eluted at 20 min, where the desired acetylated ADPr eluted around 15 min.

Preparative HPLC was performed using a Beckman Biosys 510 HPLC with column (300 Å, 5 μm, 9.4×250 mm, Vydac) at a fixed wavelength of 260 nm. For HILIC, compound separation utilized a gradient system comprising ACN (solvent A) and 10 mM NH₄OAc (solvent B) using a flow rate of 4 mL/min. The gradient was run isocratically in 20% B for 5 min followed by a linear gradient of 20-40% B over a 40-min period. For C18, compound separation utilized a gradient system comprising of H₂O with 0.05% TFA (solvent A) and ACN (with 0.02% TFA (solvent B) using a flow rate of 4 mL/min. The gradient was run isocratically in 0% B for 2 min followed by a linear gradient of 0-8% B over a 20-min period. The gradient was then increased to 100% over the next 20 min.

General Phosphatase Assay:

The following method was modified from the procedure described in Ames, 1966, Methods Enzymol. 8:115-118. The methodology employed Calf Intestine Phosphatase (CIP) to hydrolyze the phosphate ester from the ribose sugar, and the total amount of free phosphate in solution was determined using a colorimetric assay. Identification of fractions containing inorganic phosphate and/or the phosphorylated sugar followed the following steps: (1) Reaction contained 50 mM Tris (pH 7.8), 10 mM MgCl₂, 5U CIP, and 25 μL phosphate sample (200 μL final volume). Fractions containing MeOH resulted in a negligible effect on CIP activity. (2) Each tube was incubated at 37° C. for 5 min to liberate free phosphate. (3) The reactions were quenched with 600 μL of the molybdate-ascorbic acid quench solution and followed with incubation at 42° C. for 20 min. (4) A₈₂₀ was obtained to identify fractions containing the phosphorylated ribose sugar. Additional controls lacking enzyme were performed to distinguish fractions containing free inorganic phosphate from those containing the desired phosphorylated sugar.

### 2-O-Acetyl-D-ribofuranose 5-hydrogen phosphate

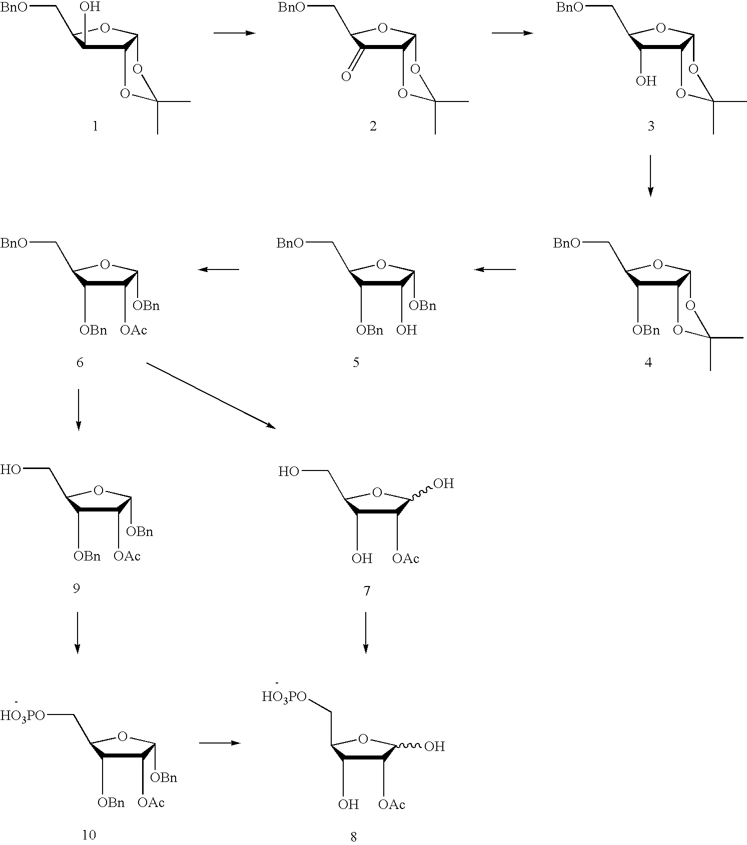

### 5-Benzyl-3-oxo-1,2-O-isopropylidene-α-D-xylofuranose (2)

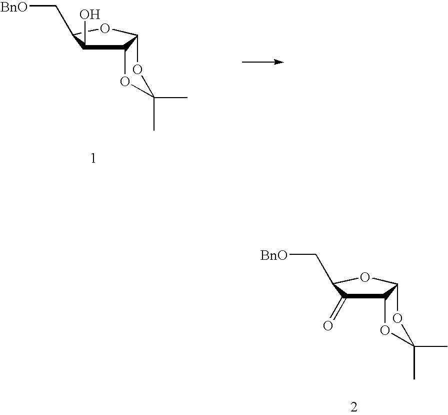

This compound was prepared according to the procedure described by Alper et al., 1998, J. Am. Chem. Soc. 120: 1965-1978. To a solution of the furanose (synthesized in above reference) (0.372 g, 1.33 mmole) in dry CH₂Cl₂ (4 mL) was added pyridinium dichromate (PDC) (0.300 g, 0.798 mmole) and acetic anhydride (0.377 mL, 3.99 mmole). The mixture was heated to reflux and maintained for 2 h. The mixture was then cooled to room temperature, diluted with Et₂O and filtered through a silica pad. The silica pad was washed with Et₂O and CH₂Cl₂). The filtrates were combined and concentrated under reduced pressure to afford the crude product. The crude product was purified via chromatography (silica gel, CHCl₃) to afford 0.278 g (75% yield) of the 5-benzyl-3-oxo-1,2-O-isopropylidene-α-D-xylofuranose. The reaction was analyzed by TLC (silica gel, CHCl₃).

### 5-Benzyl-1,2-O-isopropylidene-α-D-ribofuranose (3)

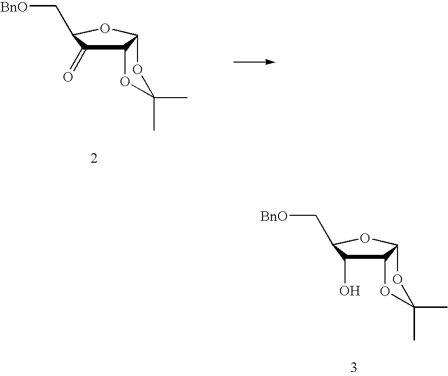

This compound was prepared according to the procedure described by Alper et al., 1998, J. Am. Chem. Soc. 120: 1965-1978. To a solution of the 5-benzyl-3-oxo-1,2-O-isopropylidene-α-D-xylofuranose in dry methanol (806 μL) was added NaBH₄. The mixture was stirred at room temperature for 1 h, then quenched with water and the mixture concentrated under reduced pressure. To the resulting residue was added EtOAc. This mixture was washed with aqueous NaHCO₃. The organic layer was dried and concentrated under reduced pressure to afford the crude product. The crude product was analyzed by TLC (silica gel, 4:4:1 hexanes/EtOAc/MeOH) and taken directly into the next step.

### 3,5-Di-benzyl-1,2-isopropylidene-α-D-ribofuranose

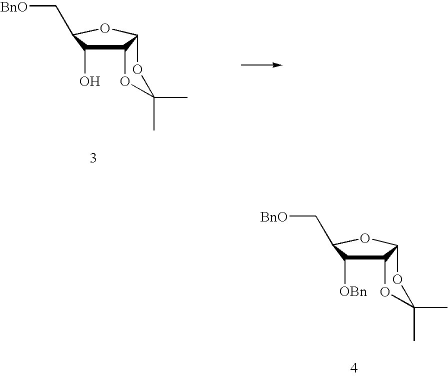

To a solution of the crude 5-benzyl-1,2-O-isopropylidene-α-D-ribofuranose (0.0556 g, 0.198 mmole) in dry DMF (1.1 mL) at 0° C., was added NaH (0.0052 g, 0.22 mmole), followed by addition of benzyl bromide (70.7 μL, 0.595 mmole). The mixture was stirred for 30 minutes, then quenched with AcOH (about 5 drops), and concentrated under reduced pressure. The resulting residue was taken up in EtOAc and the mixture washed with water, then aqueous NaHCO₃, and then brine. The organic layer was dried and concentrated under reduced pressure to afford the crude product. The crude product was purified via chromatography (silica gel, 3:1 hexanes/EtOAc) to afford 0.0476 g (65% yield over 2 steps) of 3,5-di-benzyl-1,2-isopropylidene-α-D-ribofuranose. The product was analyzed by TLC (silica gel, 2:1 hexanes/EtOAc) and the structure confirmed by ¹H NMR.

### 1,3,5-Tri-benzyl-α-D-ribofuranose (5)

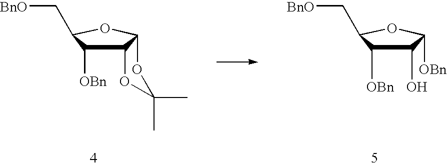

This compound was prepared according to the procedure described by Boto et al., 2003, J. Org. Chem. 68: 5310-5319. A solution of 3,5-di-benzyl-1,2-isopropylidene-α-D-ribofuranose in 1:1 TFA/H₂O (667 μL) was stirred at room temperature overnight, then concentrated under reduced pressure. EtOH was added, and the mixture concentrated under reduced pressure; this procedure was repeated twice more. To the resulting residue in dry methanol (2.7 mL) was added Bu₂SnO (0.0233 g, 0.934 mmole). The suspension was heated to reflux, and maintained at reflux for 1.5 h, then cooled to room temperature and concentrated under reduced pressure. To the crude residue in dry DMF (180 μL) was added K₂CO₃ (0.0295 g, 0.213 mmole) and benzyl bromide (21.4 μL, 0.180 mmole). The resulting mixture was stirred at room temperature overnight. The next day, the reaction mixture was filtered through Celite, and the Celite washed with CHCl₃. The filtrates were combined and concentrated under reduced pressure. The resulting residue was taken up in CHCl₃, washed with H₂O, then dried, and then concentrated under reduced pressure to afford the crude product. The crude product was purified via chromatography (silica gel, 3:1 hexanes/EtOAc) to afford 0.0073 g (26% yield) of 1,3,5-tri-benzyl-α-D-ribofuranose. The product was analyzed by TLC (silica gel, 2:1 hexanes/EtOAc) and the structure confirmed by ¹H NMR.

### 1,3,5-Tribenzyl-2-acetyl-α-D-xylofuranose (6)

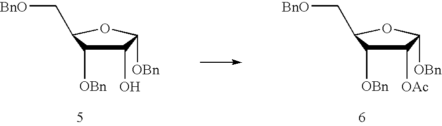

To a solution of 1,3,5-tri-benzyl-α-D-ribofuranose (0.0346 g, 0.0866 mmole) in CH₂Cl₂ (500 μL) was added pyridine (43 μL, 0.433 mmole). The solution was cooled to 0° C. and acetic anhydride (16.4 μL, 0.173 mmole) was added dropwise. After stirring at 0° C. for a few minutes, the reaction mixture was warmed to room temperature and stirred at room temperature for 1.5 h. Analysis by TLC (silica gel, 2:1 hexanes/EtOAc) indicated the reaction was incomplete. The reaction mixture was retrieved and brought up in dry pyridine (435 μL). To this solution was added acetic anhydride (139 μL, 1.47 mmole). The reaction mixture was stirred at room temperature for 2 h, then poured into ice water. The aqueous layer was extracted with CHCl₃, and the combined organic layers combined and concentrated under reduced pressure. Analysis by TLC revealed the reaction was still incomplete. The residue was resubjected to the reaction conditions overnight, and dried under vacuum. About 30 mg of crude product was obtained.

### 2-O-Acetyl-ribose (7)

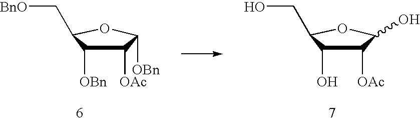

This compound was prepared according to the procedure described by Maryanoff et al., 1984, J. Am. Chem. Soc. 106: 7851-7853. To a solution of 1,3,5-tribenzyl-2-acetyl-α-D-xylofuranose in acetic acid (500 μL) was added Pd/C (about 20 mg). H₂ was introduced through a balloon and stirred for the night (24 h total). The reaction mixture was filtered through Celite, and the Celite washed with methanol. The combined filtrates were concentrated under reduced pressure. The residue was taken up in ethanol, and concentrated under reduced pressure; and then this step repeated. The product was analyzed by TLC (silica gel, 2:1 hexanes/EtOAc) and (8:1 CH₂Cl₂/MeOH) and ¹H NMR. Mass spectrum m/z: 215.3 (M+Na).

### 3,5-Di-O-benzyl-1,2-O-isopropylidene-α-D-ribofuranose (4)

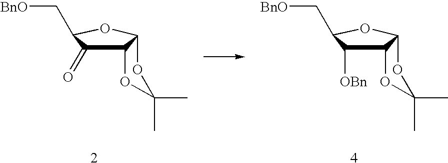

5-O-Benzyl-3-oxo-1,2-O-isopropylidene-α-D-xylofuranose (Alper et al. J. Am. Chem. Soc. 1998, 120:1965-1978) (2.00 g, 7.19 mmol) in 31 mL dry MeOH was added NaBH₄ (0.136 g, 3.60 mmol). After stirring for 1 h, the reaction was quenched with H₂O and the solvent was evaporated in vacuo. The resulting residue was re-suspended in EtOAc and washed (NaHCO₃). The organic layer was dried over Na₂SO₄ and evaporated in vacuo. To the crude ribofuranose in 40 mL dry DMF at 0° C. was added NaH (7.91 mmol), followed by BnBr (3.69 g, 21.57 mmol). After stirring for 30 min., the reaction was quenched with 500 μL AcOH and the solvent was evaporated. The residue was re-suspended in EtOAc, subjected to an aqueous workup (H₂O, NaHCO₃, EtOAc, brine), dried over Na₂SO₄, and evaporated in vacuo. Flash chromatography on silica (3:1 Hexanes/EtOAc) afforded the product (1.77 g, 67%). ¹H NMR (CDCl₃) δ 7.35-7.25 (m, 10H), 5.76 (d, J=3.7 Hz, 1H), 4.73 (d, J=11.8 Hz, 1H), 4.59-4.47 (m, 4H), 4.21-4.16 (m, 1H), 3.86 (dd, J=9.1, 4.5 Hz, 1H), 3.76 (dd, J=11.3, 1.9 Hz, 1H), 3.56 (dd, J=11.3, 3.7 Hz, 1H), 1.59 (s, 3H), 1.36 (s, 3H); ¹³C NMR (CDCl₃) δ 137.9, 137.6, 128.2, 128.1, 127.7, 127.5, 127.4, 112.6, 104.0, 77.9, 77.1, 73.2, 71.9, 68.0, 26.7, 26.4. HRMS-EI: calcd for C₂₂H₂₆O₅ (M+Na⁺), 393.1678, obsd 393.1677.

### 1,3,5-Tri-O-Benzyl-α-D-ribofuranose (5)

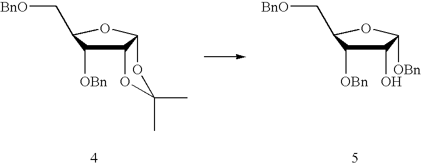

To 3,5-Di-O-Benzyl-1,2-O-isopropylidene-α-D-ribofuranose (0.656 g, 1.77 mmol) in 21 mL 1:1 dioxane/H₂O was added 1.5 mL Dowex-50WX2-100 (H⁺ form) (as a 1:1 slurry with H₂O). After the suspension was stirred at 80° C. for 22 h, the reaction was cooled to ambient temperature and the resin was filtered; the solvent was evaporated and co-stripped with EtOH (to remove trace H₂O). Upon drying in vacuo, the white solid was washed with 1:1 Et₂O/Pet Ether (with vigorous shaking). This process was repeated (typically 2-3 times) until all contaminants were removed in the organic layer to result in a white solid (0.486 g), which a portion was taken directly forward. The 3,5-Di-O-Benzyl-β-D-ribofuranose (0.322 g, 0.974 mmol) in 39 mLMeOH (HPLC grade) was added Bu₂SnO (0.339 g, 1.364 mmol). The reaction was heated at reflux for 2 h. Upon cooling to ambient temperature, the solvent was evaporated and the clear oil was dried under vacuum. To the resulting dibutylstannylene in 1.33 mL anhydrous DMF was added K₂CO₃ (0.431 g, 3.117 mmol). Benzyl bromide (0.450 g, 2.630 mmol) was added drop wise to the rapidly stirring suspension and stirred for an additional 27 h. The mixture was filtered through Celite and washed with several portions of CHCl₃. The resulting organic was washed with water, dried over Na₂SO₄, and evaporated in vacuo. Flash chromatography on silica (3:1 Hexanes/EtOAc) afforded the product (0.305 g, 74%). ¹H NMR (CDCl₃) δ 7.38-7.22 (m, 15H), 5.08 (d, J=4.6 Hz, 1H), 4.87 (d, J=12.2. Hz, 1H), 4.71 (d, J=12.0 Hz, 1H), 4.59 (ABq, J=12.0 Hz, 2H), 4.49 (ABq, J=12.1 Hz, 2H), 4.23 (dd, J=7.4, 4.1 Hz, 1H), 4.15 (ddd, J=11.5, 7.0, 4.5 Hz, 1H), 3.82 (dd, J=7.1, 3.1 Hz, 1H), 3.46 (dd, J=10.6, 4.1 Hz, 1H), 3.39 (dd, J=10.6, 4.1 Hz, 1H), 3.03 (d, J=11.4 Hz, 1H); ¹³C NMR (CDCl₃) δ 138.2, 138.1, 128.6, 128.5, 127.93, 127.90, 127.8, 127.7, 100.8, 82.3, 76.8, 73.7, 73.0, 72.2, 70.3, 69.2. HRMS-ESI: calcd for C₂₆H₂₈O₅ (M+Na⁺), 443.1834, obsd 443.1828.

### 1,3,5-Tri-O-Benzyl-2-O-acetyl-α-D-ribofuranose (6)

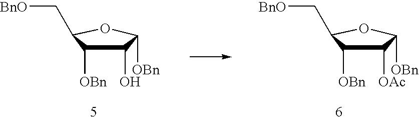

To 1,3,5-tri-O-benzyl-α-D-ribofuranose (0.208 g, 0.494 mmol) in 2.5 mL anhydrous pyridine was added acetic anhydride (0.856 g, 8.390 mmol). After stirring for 6 h, the reaction was poured into ice water and extracted with CHCl₃ (three times). The organic layers were combined, dried over Na₂SO₄, and evaporated in vacuo. Flash chromatography on silica (3:1 Hexanes/EtOAc) afforded the 1,3,5-tri-O-benzyl-2-O-acetyl-α-D-ribofuranose (0.221 g, 97%). ¹H NMR (CDCl₃) δ 7.35-7.25 (m, 15H), 5.26 (d, J=4.6 Hz, 1H), 4.94 (dd, J=7.0, 4.6 Hz, 1H), 4.86 (d, J=12.5 Hz, 1H), 4.66 (ABq, J=12.5 Hz, 2H), 4.54-4.42 (m, 3H), 4.24 (dt=q, J=8.0, 3.9 Hz, 1H), 4.05 (dd, J=6.8, 4.8 Hz, 1H), 3.49 (dd, J=10.6, 3.2 Hz, 1H), 3.36 (dd, J=10.7, 4.1 Hz, 1H), 2.16 (s, 3H); ¹³C NMR (CDCl₃) δ 170.7, 138.1, 138.1, 138.0, 128.6, 128.5, 128.4, 128.2, 128.0, 127.91, 127.88, 127.7, 99.8, 81.4, 75.6, 73.6, 73.2, 72.1, 69.5, 21.0. HRMS-EI: calcd for C₂₈H₃₀O₆ (M+Na⁺), 486.2018, obsd 486.2025.

### 1,3-Di-O-Benzyl-2-O-acetyl-α-D-ribofuranose (9)

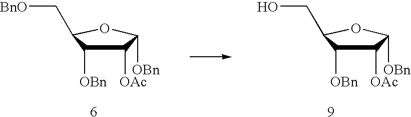

To 1,3,5-tri-O-benzyl-2-O-acetyl-α-D-ribofuranose (0.159 g, 0.3431 mmol) in 768 μL 1:1 MeOH/AcOH containing 0.08% pyridine (v/v) was added 15.3 mg Pd/C (10%). The reaction was stirred under 50 psi H₂ for 1 d. The Pd/C was filtered off through Celite and washed with MeOH. The solvent was evaporated and dried in vacuo. Flash chromatography on silica (2:1 Hexanes/EtOAc) afforded 1,3-Di-O-Benzyl-2-O-acetyl-α-D-ribofuranose (0.063 g, 49%); this reaction typically gave yields of 40-50%. Approximately 5-15% of unreacted starting material could be recovered from the reaction. ¹H NMR (CDCl₃) δ 7.34-7.25 (m, 10H), 5.23 (d, J=4.4 Hz, 1H), 4.91 (dd, J=6.7, 4.4 Hz, 1H), 4.83 (d, J=12.4 Hz, 1H), 4.66 (ABq, J=12.5 Hz, 2H), 4.49 (d, J=12.1 Hz, 1H), 4.17-4.13 (m, 1H), 4.03 (dd, J=6.7, 5.5 Hz, 1H), 3.69 (ddd, J=12.1, 4.2, 3.1 Hz, 1H), 3.42 (ddd, J=12.1, 7.8, 3.5 Hz, 1H), 2.15 (s, 3H), 1.89 (dd, J=7.7, 4.3 Hz, 1H); ¹³C NMR (CDCl₃) δ 170.7, 138.0, 137.7, 128.6, 128.5, 128.2, 127.9, 127.8, 99.8, 82.3, 75.1, 73.4, 72.2, 69.6, 62.0, 21.0. HRMS-ESI: calcd for C₂₁H₂₄O₆ (M+Na⁺), 395.1471, obsd 395.1467.

### 1,3-Di-O-Benzyl-2-O-acetyl-α-D-ribofuranose 5-hydrogen phosphate (TEA salt) (10)

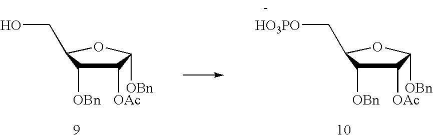

To 1,3-di-O-benzyl-2-O-acetyl-α-D-ribofuranose (0.0645 g, 0.173 mmol) in 470 μL dry THF at 0° C. was added triethylamine (0.1577 g, 1.559 mmol), followed by POCl₃ (0.0398 g, 0.260 mmol) drop wise. After stirring at 0° C. for 2 h, a few ice chips were added and stirred for an additional hour. The solvent was evaporated and the residue dried under vacuum. The desired product was purified away from free inorganic phosphate using reversed-phase flash chromatography and a step-wise gradient of MeOH (25-100% in degassed H₂O) (Kühler and Lindsten, 1983, J. Org. Chem. 48: 3589-3591; O'Neil, 1991, Synlett 9: 661-662). After identifying the desired fractions by the phosphatase assay (described above in general procedures), the fractions were combined to yield 1,3-Di-O-Benzyl-2-O-acetyl-α-D-ribofuranose 5-hydrogen phosphate (as the TEA salt) (0.0810 g, 85%). ¹H NMR (CDCl₃) δ 7.39-7.22 (m, 10H), 5.22 (d, J=4.4 Hz, 1H), 4.90 (dd, J=6.5, 4.3 Hz, 1H), 4.81 (d, J=12.7 Hz, 1H), 4.63 (s, 2H), 4.58 (d, J=12.7 Hz, 1H), 4.29-4.27 (m, 1H), 4.20 (dd, J=6.5, 4.4 Hz, 1H), 4.00 (m, 2H), 3.01 (bq, 6H), 2.09 (s, 3H), 1.26 (bt, 9H); ¹³C NMR (CDCl₃) δ 170.5, 138.5, 138.1, 128.33, 128.30, 128.1, 127.7, 127.6, 127.5, 99.7, 81.6, 75.9, 73.0, 72.4, 69.3, 64.7, 45.6, 20.9, 8.6; ³¹P NMR (CDCl₃) δ 2.2. HRMS-ESI: calcd for C₂₁H₂₄O₉P (M−H)⁻, 451.1163, obsd 451.1256.

### 2-O-Acetyl-D-ribofuranose 5-hydrogen phosphate (TEA salt) (8)

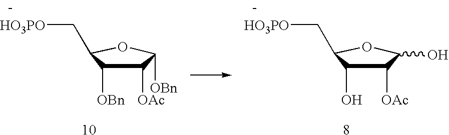

To 1,3-di-O-benzyl-2-O-acetyl-α-D-ribofuranose 5-hydrogen phosphate (as the TEA salt) (0.0608 g, 0.1098 mmol) in 4.2 mL EtOH was added 10% Pd/C (0.0417 g). The reaction was stirred under 50 psi H₂ for 2 days. The Pd/C was filtered off through Celite and washed with MeOH. The solvent was evaporated and the resulting product was dried in vacuo (0.036 g, 88%). Multiple signals were observed for the phosphorus and all ribose protons and carbons. This observation is attributable to a combination of the mixture of α and β anomers and the previously observed transesterification to the 3-position. (Jackson and Denu, 2002, J. Biol. Chem. 277: 18535-18544.) ¹H NMR (CD₃OD) δ 5.43-5.09 (m, 1H), 4.96-4.90 (m, 1H), 4.39-4.17 (m, 1H), 4.14-4.05 (m, 1H), 4.03-3.95 (m, 2H), 3.20 (bq, 6H), 2.12-2.09 (m, 3H), 1.32 (bt, 9H); ¹³C NMR (CD₃OD) δ 172.1, 171.9, 103.6, 101.0, 97.5, 96.8, 83.1, 83.0, 80.7, 80.6, 79.0, 75.6, 75.4, 74.5, 73.7, 71.7, 67.6, 67.53, 67.47, 58.3, 47.4, 21.2, 20.85, 20.77, 18.4, 9.1; ³¹P NMR (CD₃OD) δ 2.2, 1.9, 1.84, 1.78. HRMS-ESI: calcd for C₇H₁₂O₉P (M−H)⁻, 271.0224, obsd 271.0249.

### 2-N-Acetyl-2-deoxy-D-ribofuranose 5-hydrogen phosphate

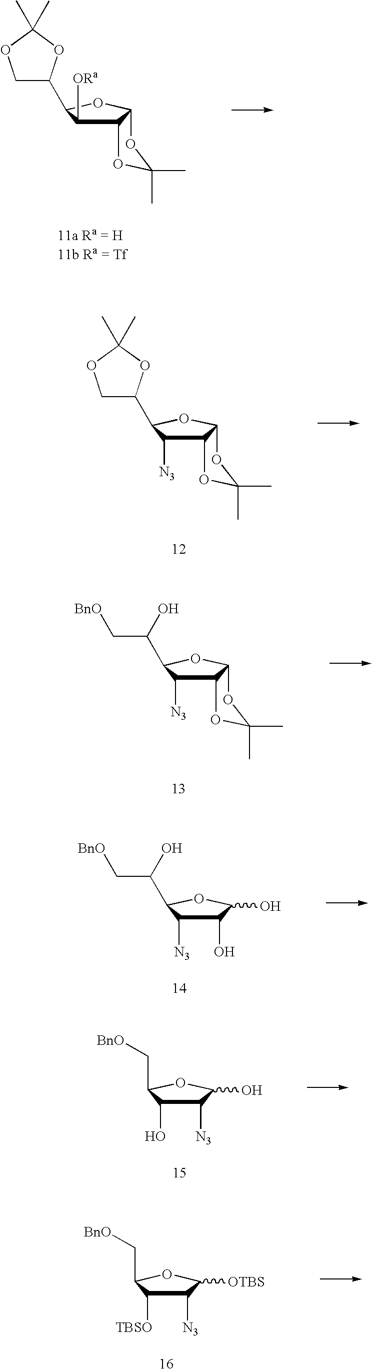

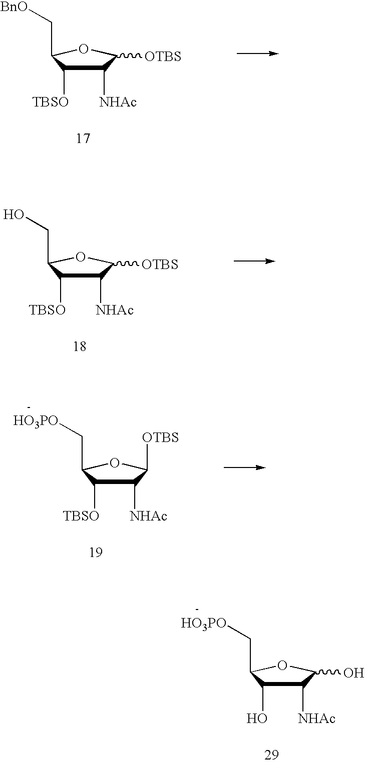

### 1,2:5,6-Di-O-isopropylidene-3-O-triflate-α-D-glucofuranose (11b)

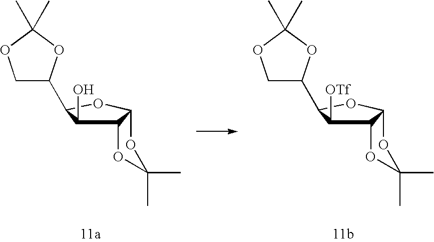

To a solution of 1,2:5,6-di-O-isopropylidene-α-D-glucofuranose (1.000 g, 3. 3844 mmole) in dry CH₂Cl₂ (80 mL) was added pyridine (1.49 mL, 13.0 mmole). The solution was cooled to −10° C. and triflic anhydride (776 μL, 4.61 mmole) was added dropwise. The reaction mixture was stirred at −10° C., and slowly warmed to 0° C. The reaction mixture was quenched, then portioned between aqueous NaHCO₃ and CH₂Cl₂. The organic layer was dried and concentrated under reduced pressure to afford the crude product. The crude product was purified by chromatography (silica gel, 4:1 hexanes/Et₂O) to afford 1.38 g (92% yield) of 1,2:5,6-di-O-isopropylidene-3-O-triflate-α-D-glucofuranose. The product was analyzed by TLC (silica gel, 2:1 hexanes/Et₂O).

### 1,2:5,6-Di-isopropylidene-3-azido-α-D-allofuranose (12)

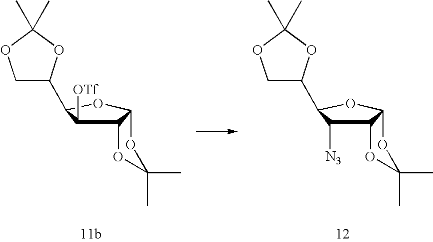

To a solution of LiF (0.330 g, 12.8 mmole) in dry DMF (6.4 mL) at 100° C. was added TMSN₃ (1.69 mL, 12.75 mmole). After stirring for 1 h, a solution of 1,2:5,6-di-O-isopropylidene-3-O-triflate-α-D-glucofuranose (1.000 g, 2.550 mmole) in dry DMF (6.4 mL) was added and the resulting reaction mixture stirred for 5 h. After cooling to room temperature, the reaction mixture was washed with aqueous NaHCO₃ (2×), and the organic layer was dried, and concentrated under reduced pressure to afford the crude product. The crude product was purified via chromatography (silica gel, 3:1 hexanes/Et₂O) to afford 0.324 g (44.6% yield) of 1,2:5,6-di-isopropylidene-3-azido-α-D-allofuranose. The reaction was analyzed by TLC (silica gel, 2:1 hexanes/Et₂O).

### 6-Benzyl-3-azido-1,2-isopropylidene-α-D-allofuranose (13)

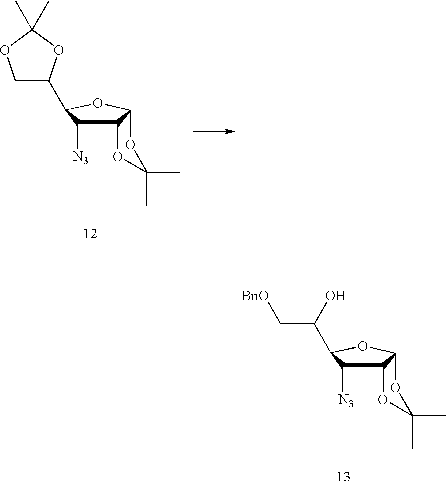

3-Azido-3-deoxy-1,2:5,6-di-O-isopropylidene-α-D-allofuranose was prepared by the procedure above or using procedures similar to the preparation of 3-azido-3-deoxy-5-O-benzyl-1,2-O-isopropylidene-α-D-ribofuranose. Spectral data was in agreement with that described in Gao et al., 2006, J. Med. Chem. 49: 2689-2702; Baer and Gan, 1991, Carbohydr. Res. 210: 233-245. 3-Azido-3-deoxy-1,2:5,6-di-O-isopropylidene-α-D-allofuranose (1.218 g, 4.272 mmol) was dissolved in 17 mL 70% aqueous AcOH and stirred overnight. The solvent was evaporated in vacuo and co-stripped with EtOH three times to remove all traces of acid; the resulting oil was dried under vacuum. To the crude furanose in 23 mL dry toluene was added Bu₂SnO (1.276 g, 5.126 mmol); the reaction was then refluxed overnight with azeotropic removal of water. The Dean-Stark trap was then removed and replaced with a standard reflux condenser. BnBr (0.71 mL, 5.981 mmol) and Bu₄NBr (0.689 g, 2.136 mmol) were added and stirred at 110° C. for an additional 6 h. Upon cooling to ambient temperature, the solvent was evaporated and the residue was dried in vacuo. Flash chromatography on silica (20→50% EtOAc in Hexanes) provided 6-Benzyl-3-azido-1,2-isopropylidene-α-D-allofuranose (0.935 g, 65%). ¹H NMR (CDCl₃) δ 7.34 (m, 5H), 5.76 (d, J=3.6 Hz, 1H), 4.69 (dd, J=4.5, 4.0 Hz, 1H), 4.56 (s, 2H), 4.13 (dd, J=9.1, 4.3 Hz, 1H), 4.08-4.03 (m, 1H), 3.66-3.54 (m, 3H), 2.82 (d, J=3.3 Hz, 1H), 1.56 (s, 3H), 1.35 (s, 3H); ¹³C NMR (CDCl₃) δ 137.7, 128.5, 127.94, 127.92, 113.2, 104.1, 80.8, 77.9, 73.5, 70.7, 70.0, 60.5, 26.6, 26.5. HRMS-ESI: calcd for C₁₆H₂₁N₃O₅ (M+Na⁺), 358.1379, obsd 358.1396.

### 5-Benzyl-2-azido-2-deoxy-ribose (15)

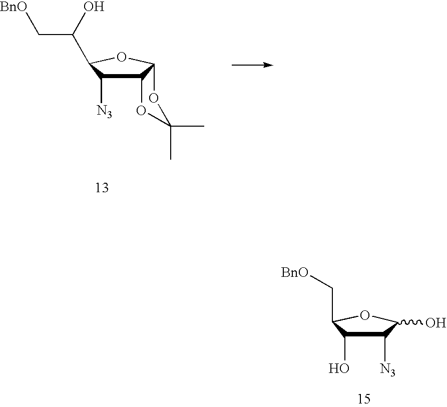

To a solution of 6-benzyl-3-azido-1,2-isopropylidne-α-D-allofuranose (0.1164 g, 0.3473 mmole) in 1:1 dioxane/H₂O (1.55 mL) was added Dowex-50 (H+) resin (120 μL of a 1:1 slurry). The reaction mixture was heated at 80° C. for 20 h. After cooling to room temperature, the mixture was filtered, and the filter cake washed with dioxane (about 220 μL). To the combined filtrates was added a mixture of NaIO₄ (0.0780 g, 0.3647 mmole) in water (590 μL). After stirring for 1 h, an additional aliquot of NaIO₄ (23.6 mg, 0.1105 mmole) in water (180 μL) was added. After stirring for 1 h, NaHCO₃ (0.0350 g, 0.417 mmole) was added and the mixture stirred overnight. The reaction mixture was filtered through Celite, and the filter cake washed with EtOAc. The combined filtrates were concentrated under reduced pressure, then taken up in EtOAc, washed with water, dried, and concentrated under reduced pressure to afford 84.2 mg of the crude product which was taken into the next step. The reaction was analyzed by TLC (silica gel, 2:1 CHCl₃/EtOAc).

### 5-Benzyl-2-azido-2-deoxy-1,3-OTBS ribose (16)

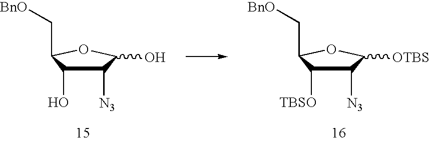

To a solution of 5-benzyl-2-azido-ribose in dry DMF (1.66 mL) was added imidazole (0.1081 g, 1.588 mmole) and t-butyldimethysilyl chloride (0.1436, 0.9528 mmole). After stirring for 5 h, the reaction mixture was partitioned between EtOAc and aqueous NH₄Cl. The organic layer was washed with brine, dried and concentrated under reduced pressure to afford the crude product. The crude product was purified via chromatography (silica gel, gradient hexanes→10% EtOAc/hexanes) to afford 0.0795 g (46.5% yield) of 5-benzyl-2-azido-1,3-OTBS ribose. The reaction was analyzed by TLC (silica gel, 6:1 hexanes/EtOAc).

### 2-Azido-2-deoxy-5-O-benzyl-1,3-O-bis-(tert-butyidimethylsilyl)-D-ribofuranose (16)

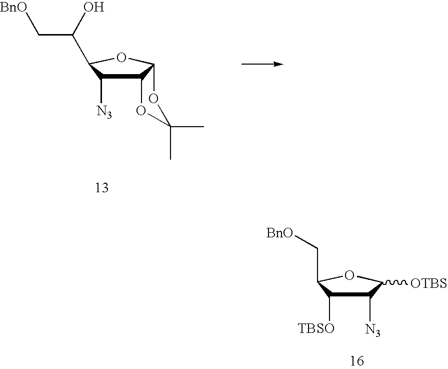

To 3-Azido-3-deoxy-6-O-benzyl-1,2-O-isopropylidene-α-D-allofuranose (0.558 g, 1.665 mmol) in 7.5 mL 1:1 dioxane/H₂O was added 650 μL Dowex-50WX2-100 (H⁺ form) (as a 1:1 slurry with H₂O). After stirring at 80° C. for 20 h, the reaction was cooled; the resin was filtered and washed with 1.1 mL dioxane. NaIO₄ (0.374 g, 1.749 mmol) in 2.8 mL H₂O was added slowly to the stirring solution and stirred for one hour. Additional NaIO₄ (0.119 g, 0.556 mmol) in 860 μL H₂O was added and stirred for an additional hour. Finally, NaHCO₃ (0.168 g, 1.999 mmol) was added in small portions and the mixture was stirred overnight. The resulting suspension was filtered through Celite and washed with EtOAc. The combined filtrates were concentrated, brought up in EtOAc, and washed with H₂O; the organic was dried over Na₂SO₄ and evaporated in vacuo. The material was purified on silica (3:1 CHCl₃/EtOAc) to afford 2-azido-2-deoxy-5-O-benzyl-D-ribofuranose (0.267 g, 1.007 mmol) and taken directly forward. The diol was brought up in 5.2 mL dry DMF and imidazole (0.343 g, 5.04 mmol) and TBSCl (0.455 g, 3.02 mmol) were added. After stirring overnight, the reaction was washed (NH₄Cl twice, EtOAc, brine), dried over Na₂SO₄ and evaporated in vacuo. Flash chromatography on silica (0→5% EtOAc in Hexanes) afforded 2-Azido-2-deoxy-5-O-benzyl-1,3-O-bis-(tert-butyldimethylsilyl)-D-ribofuranose as a mixture of α and β anomers (1:6 ratio) (0.492 g, 60%). Signals for the two anomers could be discerned by ¹H NMR (CDCl₃): α-¹H NMR (CDCl₃) δ 7.34-7.28 (m, 5H), 5.52 (d, J=4.3 Hz, 1H), 4.53 (ABq, J=12.1 Hz, 2H), 4.29 (dd, J=7.0, 3.3 Hz, 1H), 4.19 (dd, J=6.4, 3.5 Hz, 1H), 3.57-3.51 (m, 2H), 3.04 (dd, J=6.8, 4.3 Hz, 1H), 0.94 (s, 9H), 0.89 (s, 9H), 0.17 (s, 3H), 0.16 (s, 3H), 0.12 (s, 3H), 0.01 (s, 3H); ¹³C NMR (CDCl₃) δ 138.3, 138.1, 128.6, 128.5, 127.9, 127.7, 100.5, 98.9, 85.0, 82.5, 73.8, 73.7, 73.5, 71.2, 69.5, 68.9, 62.4, 26.0, 25.9, 25.8, 18.2, 18.1, 18.0, −4.1, −4.3, −4.5, −4.7, −4.88, −4.91, −5.1. β-δ 7.34-7.27 (m, 5H), 5.20 (d, J=1.4 Hz, 1H), 4.57 (s, 2H), 4.45 (dd, J=6.3, 5.0 Hz, 1H), 4.06 (td, J=6.2, 3.5 Hz, 1H), 3.62 (dd, J=10.5, 3.5 Hz, 1H), 3.55-3.49 (m, 2H), 0.90 (s, 9H), 0.87 (s, 9H), 0.12 (s, 3H), 0.11 (s, 3H), 0.09 (s, 3H), 0.08 (s, 3H); HRMS-EI: calcd for C₂₄H₄₃N₃O₄Si₂ (M+Na⁺), 516.2690, obsd 516.2696.

### 2-N-acetyl-2-deoxy-5-O-Benzyl-1,3-O-bis-(tert-butyldimethylsilyl)-D-ribofuranose (17)

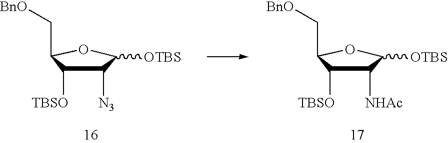

To 2-Azido-2-deoxy-5-O-Benzyl-1,3-O-bis-(tert-butyldimethylsilyl)-D-ribofuranose (0.492 g, 0.998 mmol) in 14.6 mL 4:2:1:1 pyridine/7N methanolic NH₃/MeOH/H₂O was added PPh₃ (0.707 g, 2.69 mmol). After stirring the reaction overnight, the solvent was evaporated off and co-stripped with EtOH (2×) to remove trace water. After drying under vacuum, the resulting solid was dissolved in 4.3 mL dry CH₂Cl₂ and pyridine was added (0.237 g, 2.993 mmol) and cooled to 0° C. Acetic anhydride (0.122 g, 1.197 mmol) was added drop wise and the reaction was allowed to warm to rt. After stirring for 2 h, the reaction was washed (H₂O, CH₂Cl₂), dried over Na₂SO₄ and evaporated in vacuo. Flash chromatography on silica (3:1 Hexanes/EtOAc) afforded 2-N-acetyl-2-deoxy-5-O-Benzyl-1,3-O-bis-(tert-butyldimethylsilyl)-D-ribofuranose as a mixture of α and β-anomers (1:6 ratio) (0.427 g, 84%). Signals for the two anomers could be discerned by ¹H NMR (CDCl₃): α-δ 7.34-7.27 (m, 5H), 5.95 (bd, 1H), 5.38 (d, J=4.3 Hz, 1H), 4.52 (m, 2H), 4.37-4.34 (m, 1H), 4.16-4.14 (m, 1H), 4.14-4.12 (m, 1H), 3.54-3.50 (m, 2H), 1.99 (s, 3H), 0.90 (s, 18H), 0.12 (s, 3H), 0.10 (s, 3H), −0.02 (s, 3H), −0.04 (s, 3H); β-δ 7.34-7.27 (m, 5H), 5.99 (bd, 1H), 5.32 (s, 1H), 4.58 (m, 2H), 4.27 (t, J=5.6 Hz, 1H), 4.02 (t, J=5.6 Hz, 1H), 4.00-3.98 (m, 1H), 3.65-3.56 (m, 2H), 2.00 (s, 3H), 0.88 (s, 18H), 0.13 (s, 3H), 0.09 (s, 3H), 0.06 (s, 6H); ¹³C NMR (CDCl₃) δ 170.3, 169.2, 138.2, 138.0, 128.5, 128.4, 128.0, 127.9, 127.7, 101.8, 96.8, 85.3, 83.6, 73.6, 73.5, 72.4, 72.3, 71.5, 69.9, 59.6, 54.3, 25.8, 23.4, 23.3, 18.1, 18.0, −4.1, −4.4, −4.6, −4.9, −5.0, −5.1, −5.3. HRMS-EI: calcd for C₂₆H₄₇NO₅Si₂ (M+Na⁺), 532.2891, obsd 532.2885.

### 2-N-Acetyl-2-deoxy-1,3-O-bis-(tert-butyidimethylsilyl)-D-ribofuranose (18)

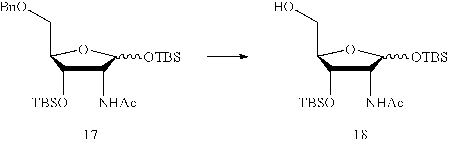

To a solution of 2-N-acetyl-2-deoxy-5-O-Benzyl-1,3-O-bis-(tert-butyldimethylsilyl)-D-ribofuranose (0.389 g, 0.0763 mmole) in MeOH (3.2 mL) was added Pd/C (about 30 mg). The resulting mixture was subjected to 50 psi H₂ for 70 h. The reaction mixture was filtered through Celite, and the filter cake washed. The combined filtrates were concentrated under reduced pressure to afford the crude product. The crude product was purified via chromatography (silica gel, gradient 2:1→1:2 hexanes/EtOAc) to afford 0.276 g (86.5% yield) of 2-N-acetyl-1,3-OTBS-ribose. The product was analyzed by TLC (silica gel, 1:1 hexanes/EtOAc) and ¹H NMR.

### 2-N-Acetyl-2-deoxy-1,3-O-bis-(tert-butyidimethylsilyl)-D-ribofuranose (18)

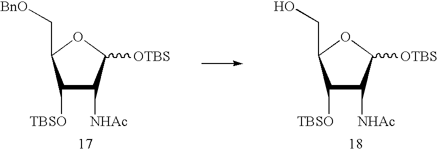

To 2-N-acetyl-2-deoxy-5-O-Benzyl-1,3-O-bis-(tert-butyldimethylsilyl)-D-ribofuranose (0.248 g, 0.486 mmol) in 20.3 mL EtOH was added 10% Pd/C (0.160 g). The reaction was stirred under 50 psi H₂ for 2 days. The Pd/C was filtered off through Celite and washed with EtOH. The solvent was evaporated and dried in vacuo. The two anomers of 2-N-acetyl-2-deoxy-1,3-O-bis-(tert-butyldimethylsilyl)-D-ribofuranose were separable by column chromatography on silica using a gradient from 2:1 Hexanes/EtOAc→2:1 EtOAc/Hexanes: (β-anomer: 0.143 g, α-anomer: 0.030 g, 85% overall). α: ¹H NMR (CDCl₃) δ 5.97 (bd, J=8.8 Hz, 1H), 5.39 (d, J=4.3 Hz, 1H), 4.27 (ddd, J=8.8, 7.3, 4.3 Hz, 1H), 4.14 (dd, J=7.5, 2.8 Hz, 1H), 4.08 (dt, J=4, 2.8 Hz, 1H), 3.78 (ddd, J=11.9, 5.2, 3.3 Hz, 1H), 3.63 (ddd, J=11.9, 7.6, 4.2 Hz, 1H), 2.00 (s, 3H), 1.77 (dd, J=7.7, 5.2 Hz, 1H), 0.91 (s, 9H), 0.90 (s, 9H), 0.12 (s, 3H), 0.10 (s, 3H), 0.06 (s, 3H), 0.05 (s, 3H); ¹³C NMR (CDCl₃) δ 169.5, 96.9, 86.7, 70.8, 62.8, 54.7, 25.8, 23.3, 18.1, 18.0, −4.2, −4.5, −4.9, −5.3. β: ¹H NMR (CDCl₃) δ 6.07 (bd, J=4 Hz, 1H), 5.37 (s, 1H), 4.74 (t, J=6.2 Hz, 1H), 4.00 (dt, J=6, 2.5 Hz, 1H), 3.91 (dd, J=6, 4.8 Hz, 1H), 3.80 (dt, J=12, 2.1 Hz, 1H), 3.58 (dd, J=12.2, 10, 2.6 Hz, 1H), 2.59 (dd, J=10, 2.6 Hz, 1H), 2.03 (s, 3H), 0.92 (s, 9H), 0.91 (s, 9H), 0.20 (s, 3H), 0.17 (s, 3H), 0.12 (s, 3H), 0.10 (s, 3H); ¹³C NMR (CDCl₃) δ 170.7, 101.3, 85.8, 69.5, 61.8, 60.3, 25.81, 25.76, 23.3, 18.1, 18.0, −4.7, −4.8, −4.9, −5.0. HRMS-ESI: calcd for C₁₉H₄₁NO₅Si₂ (M+Na⁺), 442.2421, obsd 442.2409.

### 2-N-Acetyl-2-deoxy-1,3-O-bis-(tert-butyidimethylsilyl)-β-D-ribofuranose 5-hydrogen phosphate (19)

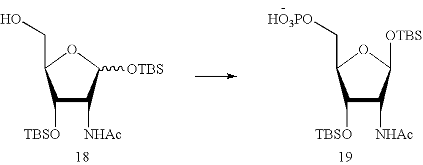

To a solution of pyridine (37.0 μL, 0.458 mmole) in acetonitrile (28 μL) at 0° C. was added POCl₃ (8.0 μL, 0.0858 mmole). This solution was added to a solution of 2-N-Acetyl-2-deoxy-1,3-O-bis-(tert-butyldimethylsilyl)-β-D-ribofuranose (0.0240 g, 0.0572 mmole) in acetonitrile (28 μL). The resulting mixture was stirred at 0° C. for 2 h. Water (200 μL) was added, and the mixture was stirred for another 45 min. The reaction mixture was concentrated under reduced pressure. The residue was dissolved in water, and the pH adjusted to about 7 with 1M NaOH. The resulting solution was lyophilized.

### 2-N-Acetyl-2-deoxy-1,3-O-bis-(tert-butyidimethylsilyl)-β-D-ribofuranose 5-hydrogen phosphate (TEA salt) (19)

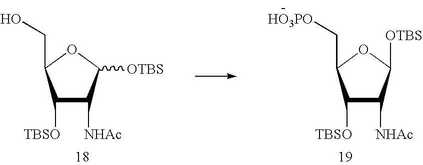

To 2-N-Acetyl-2-deoxy-1,3-O-bis-(tert-butyldimethylsilyl)-β-D-ribofuranose (0.096 g, 0.229 mmol) in 620 μL dry THF at 0° C. was added triethylamine (0.208 g, 2.06 mmol), followed by POCl₃ (0.053 g, 0.343 mmol) drop wise. After stirring cold for 2 h, a few ice chips were added and stirred for an additional hour. The solvent was evaporated and the residue dried in vacuo. The desired product was purified from free inorganic phosphate using reversed-phase flash chromatography utilizing a step-wise gradient of MeOH (25-100% in degassed H₂O) (Kuhler and Lindsten, 1983, J. Org. Chem. 48: 3589-3591; O'Neil, 1991, Synlett 9: 661-662). After identifying the desired fractions by the phosphatase assay (described above in general procedures), the fractions were combined to yield 2-N-Acetyl-2-deoxy-1,3-O-bis-(tert-butyldimethylsilyl)-β-D-ribofuranose 5-hydrogen phosphate as the TEA salt (0.128 g, 93%). ¹H NMR (CDCl₃) δ6.07 (bd, 1H), 5.16 (d, J=1.9 Hz, 1H), 4.38-4.33 (m, 1H), 4.03-3.96 (m, 2H), 3.92-3.86 (m, 2H), 3.01 (bq, 6H), 1.94 (s, 3H), 1.26 (bt, 9H), 0.84 (s, 9H), 0.82 (s, 9H), 0.09 (s, 3H), 0.05 (s, 3H), 0.04 (s, 3H), 0.01 (s, 3H); ¹³C NMR (CDCl₃) δ 170.0, 101.8, 84.0, 72.5, 66.6, 59.2, 45.5, 25.9, 25.8, 23.3, 18.1, 18.0, 8.7, −3.8, −4.3, −4.6, −5.1; ³¹P NMR (CDCl₃) δ 2.1. HRMS-ESI: calcd for C₁₉H₄₂NO₈PSi₂ (M−H)⁻, 498.2114, obsd 498.2106.

### 2-N-Acetyl-2-deoxy-D-ribofuranose 5-hydrogen phosphate (TEA salt) (29)

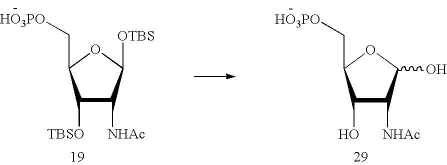

To 2-N-Acetyl-2-deoxy-1,3-O-bis-(tert-butyldimethylsilyl)-β-D-ribofuranose 5-hydrogen phosphate (TEA salt) (0.0200 g, 0.033 mmol) in 300 μL ACN was added 165 μL Dowex-50WX2-100 (H⁺ form) (as a 1:1 slurry with H₂O). The reaction was stirred for 2 d. The reaction was passed over Dowex-50WX8 (TEA form) and the solvent was evaporated and the residue dried under vacuum. The desired product was passed over a reversed-phase silica plug and washed with H₂O (5 mL). The solvent was evaporated to yield 2-N-acetyl-2-deoxy-D-ribofuranose 5-hydrogen phosphate as the TEA salt (0.0120 g, 97%). A combination of N-Acetyl rotomers and α and β anomers (3:5 ratio) was observed by NMR. ¹H NMR [distinct signals for the anomeric position (C-1) and acetyl could be discerned] (D₂O) δ 5.46 (m, 0.62H), 5.26 (m, 0.38H), 4.38-4.29 (m, 1H), 4.27-4.24 (m, 1H), 4.20-4.08 (m, 1H), 4.0-3.92 (m, 2H), 3.17 (q, J=7.3 Hz, 6H), 2.06, 2.04 (s, s, 3H), 1.25 (t, J=7.3 Hz, 9H); ¹³C NMR [multiple signals were observed for the combination of rotomers/anomers of the ribofuranose carbons and the acetyl group only] (D₂O) δ 174.8, 174.5, 174.2, 100.1, 95.8, 95.0, 84.3, 84.1, 82.9, 82.7, 70.1, 69.7, 65.8, 65.7, 65.2, 65.1, 57.9, 54.2, 46.8, 22.0, 21.9, 8.4; ³¹P NMR (CDCl₃) δ 1.0. HRMS-ESI: calcd for C₇H₁₄NO₈P (M−H)⁻, 270.0384, obsd 270.0415.

### 3-N-Acetyl-3-deoxy-D-ribofuranose 5-hydrogen phosphate

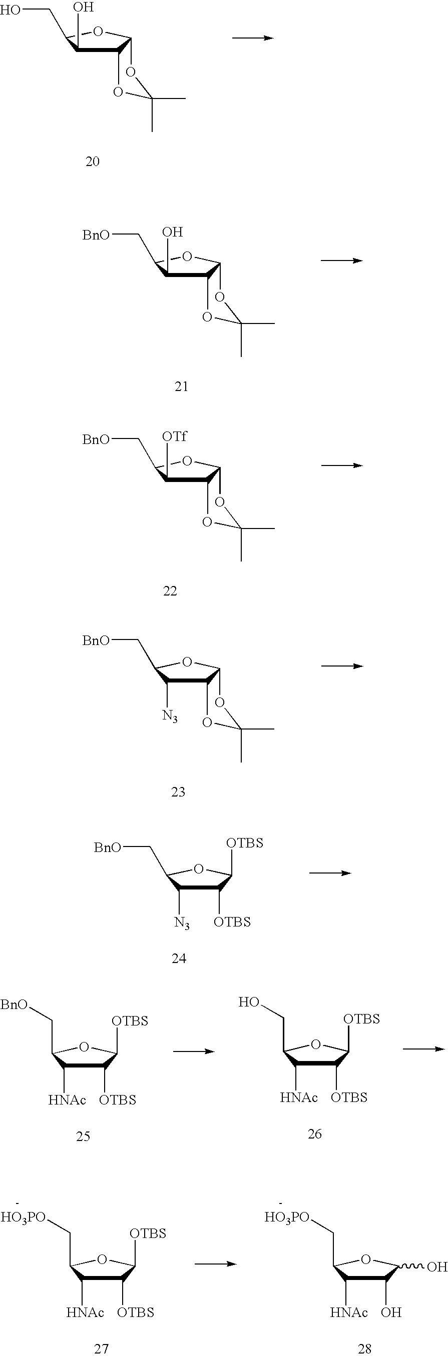

### 5-Benzyl-1,2-O-isopropylidene-α-D-xylofuranose (21)

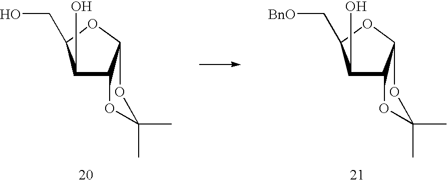

To a solution of the furanose (3.000 g, 15.77 mmole) in dry toluene (85 mL) was added Bu₂SnO (4.124 g, 16, 57 mmole). The reaction flask was equipped with a Dean-Stark apparatus, and the reaction mixture heated to reflux and maintained at reflux with removal of water overnight. The temperature was lowered to about 110° C., and benzyl bromide (3.11 mL, 23.7 mmole) and Bu₄NBr (1.526 g, 4.734 mmole) were added. The resulting reaction mixture was stirred for 6 h. After cooling to room temperature, the reaction mixture was diluted with EtOAc and NaHCO₃, filtered through Celite, and washed. The combined filtrates were portioned between NaHCO₃, and the organic layer washed with brine, dried, and concentrated under reduced pressure to afford the crude product. The crude product was purified via chromatography (silica gel, 3:2 hexanes/EtOAc) to afford 2.350 g (53.2% yield) of 5-benzyl-1,2-O-isopropylidne-α-D-xylofuranose. The reaction was analyzed by TLC (4:4:1 hexanes/EtOAc/MeOH).

### 5-O-Benzyl-3-triflyl-1,2-O-isopropylidene-α-D-xylofuranose (22)

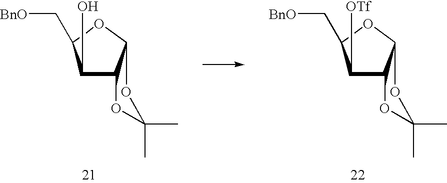

To a solution of 5-O-benzyl-1,2-O-isopropylidne-α-D-xylofuranose (0.110 g, 0.393 mmole) in anhydrous CH₂Cl₂ (8.2 mL) at −15° C., was added triflic anhydride (79.3 μL, 0.471 mmole) dropwise. The reaction mixture was warmed to 0° C. over 1 h, and then quenched with NaHCO₃. The reaction mixture was washed with aqueous NaHCO₃, dried and concentrated under reduced pressure to afford the crude product. The crude product was purified via chromatography (silica gel, 4:1 hexanes/Et₂O) to afford 0.136 g (84% yield) of 5-benzyl-3-triflate-1,2-O-isopropylidene-α-D-xylofuranose. The reaction was analyzed by TLC (silica gel, 2:1 hexanes/Et₂O).

### 5-O-Benzyl-3-O-triflyl-1,2-O-isopropylidene-α-D-xylofuranose (22)

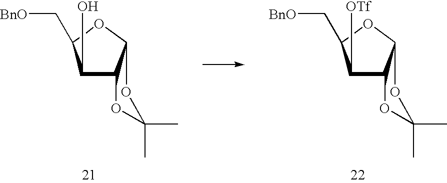

To 5-O-benzyl-1,2-O-isopropylidene-α-D-xylofuranose (Alper et al., 1998, J. Am. Chem. Soc. 120:1965-1978) (2.00 g, 7.14 mmol) in 150 mL dry CH₂Cl₂ at −10° C. was added pyridine (2.26 mL, 27.85 mmol). Trifluoro-methanesulfonic anhydride (1.44 mL, 8.57 mmol) was added drop wise and the reaction was slowly warmed to 0° C. over an hour. Saturated NaHCO₃ was added to quench the reaction and followed by aqueous workup (NaHCO₃, CH₂Cl₂). The organic layer was dried over Na₂SO₄ and dried in vacuo. Flash chromatography on silica (4:1 Hexanes/EtOAc) yielded 5-O-Benzyl-3-O-triflyl-1,2-O-isopropylidene-α-D-xylofuranose (2.58 g, 87%). ¹H NMR (CDCl₃) δ 7.36-7.30 (m, 5H), 5.99 (d, J=3.7 Hz, 1H), 5.28 (d, J=2.6 Hz, 1H), 4.73 (d, J=3.9 Hz, 1H), 4.55 (ABq, J=11.6 Hz, 2H), 4.54-4.49 (m, 1H), 3.78 (dd, J=9.6, 5.8 Hz, 1H), 3.67 (dd, J=9.6, 7.6 Hz, 1H), 1.49 (s, 3H), 1.32 (s, 3H); ¹³C NMR (CDCl₃) δ 137.5, 128.7, 128.21, 128.17, 113.2, 104.8, 88.4, 83.2, 80.1, 77.5, 74.1, 66.2, 26.7, 26.5. HRMS-EI: calcd for C₁₃H₁₉F₃O₈S (M+Na⁺), 435.0701, obsd 435.0701.

### 3-Azido-3-deoxy-5-O-benzyl-1,2-O-isopropylidene-α-D-ribofuranose (23)

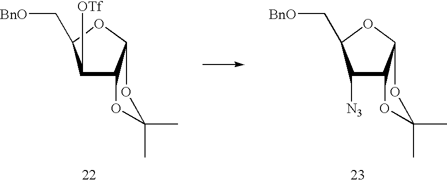

To LiF (0.570 g, 21.97 mmol) in 15.7 mL dry DMF at 100° C. was added TMSN₃ (2.531 g, 21.97 mmol). After stirring for 1 h, 5-O-Benzyl-3-O-triflyl-1,2-O-isopropylidene-α-D-xylofuranose (2.58 g, 6.28 mmol) in 15.7 mL dry DMF was added and the reaction was stirred for an additional 5 h. Upon cooling to ambient temperature, the reaction was washed (NaHCO₃ twice, CHCl₃), dried over Na₂SO₄ and dried in vacuo. Flash chromatography on silica (3:1 Hexanes/Et₂O) provided the 3-Azido-3-deoxy-5-O-benzyl-1,2-O-isopropylidene-α-D-ribofuranose (0.843 g, 44%). ¹H NMR (CDCl₃) δ 7.33-7.27 (m, 5H), 5.79 (d, J=3.8 Hz, 1H), 4.67 (dd, J=4.3, 3.9 Hz, 1H), 4.58 (ABq, J=12.1 Hz, 2H), 4.20-4.15 (m, 1H), 3.78 (dd, J=11.3, 2.3 Hz, 1H), 3.61 (dd, J=11.3, 3.7 Hz, 1H), 3.56 (dd, J=9.5, 4.7 Hz, 1H), 1.55 (s, 3H), 1.34 (s, 3H); ¹³C NMR (CDCl₃) δ 137.3, 128.4, 127.8, 127.7, 127.6, 113.0, 104.2, 79.9, 77.3, 73.7, 67.8, 60.5, 26.41, 26.39. HRMS-EI: calcd for C₁₅H₁₉N₃O₄ (M+Na⁺), 328.1273, obsd 328.1285.

### 3-Azido-3-deoxy-5-O-benzyl-1,2-O-bis-(tert-butyidimethylsilyl)-β-D-ribofuranose (24)

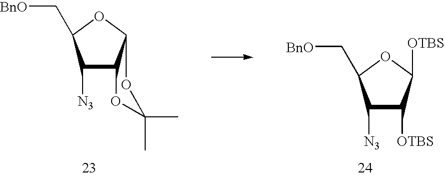

A solution of 3-Azido-3-deoxy-5-O-benzyl-1,2-O-isopropylidene-α-D-ribofuranose (0.123 g, 0.403 mmole) in 2:1:1 TFA/dioxane/water (6.2 mL) was stirred at room temperature over the weekend, then concentrated under reduced pressure. EtOH was added, and the solution concentrated under reduced pressure (3×). The resulting residue was dried under vacuum. To a solution of the residue in dry DMF (4.2 mL) was added imidazole (0.137 g, 2.02 mmole) and t-butyldimethylsilyl chloride (0.152 g, 1.01 mmole). The resulting reaction mixture was stirred at room temperature overnight, then portioned between aqueous NH₄Cl and EtOAc. The organic layer was washed with aqueous NH₄Cl and brine, dried, and concentrated under reduced pressure to afford the crude product. The crude product was purified via chromatography (silica gel, gradient hexanes→20:1 hexanes/EtOAc) to afford 0.134 g, (67% yield) of 5-benzyl-3-azido-1,2-OTBS ribose. The product was analyzed by TLC (silica gel, 4:1 hexanes/EtOAc) and ¹H NMR.

### 3-Azido-3-deoxy-5-O-benzyl-1,2-O-bis-(tert-butyidimethylsilyl)-β-D-ribofuranose (24)

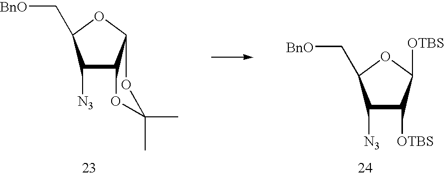

To 3-Azido-3-deoxy-5-O-benzyl-1,2-O-isopropylidene-α-D-ribofuranose (0.843 g, 2.763 mmol) in 12.5 mL 1:1 dioxane/H₂O was added 1 mL Dowex-Dowex-50WX2-100 (H⁺ form) (as a 1:1 slurry with H₂O). After the suspension was stirred at 80° C. for 22 h, the reaction was cooled to ambient temperature and the resin was filtered off and the solvent was evaporated off and co-stripped with EtOH (to remove trace H₂O). Upon drying completely in vacuo, the crude material was brought up in 25 mL dry DMF; imidazole (0.940 g, 13.81 mmol) and TBSCl (1.250 g, 8.288 mmol) were added. After stirring overnight, the reaction was washed (NH₄Cl twice, EtOAc, brine), dried over Na₂SO₄ and evaporated in vacuo to dryness. Flash chromatography on silica (0→5% EtOAc in Hexanes) afforded 3-Azido-3-deoxy-5-O-benzyl-1,2-O-bis-(tert-butyldimethylsilyl)-β-D-ribofuranose (0.877 g, 64%) predominantly as the β-anomer (signals corresponding to α-hydrogen at C1 were <1%). ¹H NMR (CDCl₃) 7.35-7.27 (m, 5H), 5.12 (s, 1H), 4.59 (s, 2H), 4.32-4.26 (m, 1H), 4.05 (d, J=4.2 Hz, 1H), 3.67-3.56 (m, 3H), 0.92 (s, 9H), 0.85 (s, 9H), 0.15 (s, 3H), 0.12 (s, 3H), 0.09 (s, 3H), 0.07 (s, 3H); ¹³C NMR (CDCl₃) δ 138.3, 128.5, 127.9, 127.8, 102.8, 79.4, 78.7, 73.7, 72.1, 62.3, 25.9, 25.8, 18.3, 18.0, −4.1, −4.70, −4.73, −5.07. HRMS-EI: calcd for C₂₄H₄₃N₃O₄Si₂ (M+Na⁺), 516.2690, obsd 516.2676.

### 3-N-Acetyl-3-deoxy-5-O-benzyl-1,2-O-bis-(tert-butyidimethylsilyl)-β-D-ribofuranose (25)

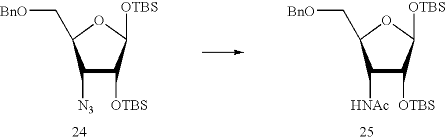

To 3-Azido-3-deoxy-5-O-benzyl-1,2-O-bis-(tert-butyldimethylsilyl)-β-D-ribofuranose (0.688 g, 1.395 mmol) in 20.6 mL 4:2:1:1 pyridine/7N methanolic NH₃/MeOH/H₂O was added PPh₃ (0.988 g, 3.77 mmol). After stirring the reaction overnight, the solvent was evaporated off and co-stripped with EtOH (×2) to remove trace water. After drying under vacuum, the resulting solid was brought up in 5.5 mL dry CH₂Cl₂ and pyridine (0.331 g, 4.184 mmol) was added and cooled to 0° C. Acetic anhydride (0.171 g, 1.674 mmol) was added drop wise and the reaction was then allowed to warm to rt. After stirring for 2 h, the reaction was washed (H₂O, CH₂Cl₂), dried over Na₂SO₄ and evaporated in vacuo. Flash chromatography on silica (2:1 Hexanes/EtOAc) yielded 3-N-Acetyl-3-deoxy-5-O-benzyl-1,2-O-bis-(tert-butyldimethylsilyl)-β-D-ribofuranose (0.669 g, 94%). ¹H NMR (CDCl₃) δ 7.36-7.21 (m, 5H), 5.80 (bd, J=8.9 Hz, 1H), 5.14 (s, 1H), 4.56 (s, 2H), 4.47 (td, J=8.9, 4.5 Hz, 1H), 4.01 (td, J=8.1, 3.0 Hz, 1H), 3.93 (d, J=4.5 Hz, 1H), 3.71 (dd, J=10.3, 2.9 Hz, 1H), 3.58 (dd, J=10.3, 8.1 Hz, 1H), 1.96 (s, 3H), 0.91 (s, 9H), 0.86 (s, 9H), 0.09 (s, 9H), 0.06 (s, 3H); ¹³C NMR (CDCl₃) δ 169.6, 138.4, 128.4, 127.9, 127.6, 102.8, 81.8, 77.8, 73.6, 73.5, 52.0, 25.8, 25.7, 23.4, 18.2, 17.9, −4.1, −4.5, −4.9, −5.2. HRMS-ESI: calcd for C₂₆H₄₇NO₅Si₂ (M+Na⁺), 532.2891, obsd 532.2902.

### 3-N-Acetyl-3-deoxy-1,2-O-bis-(tert-butyidimethylsilyl)-β-D-ribofuranose (26)

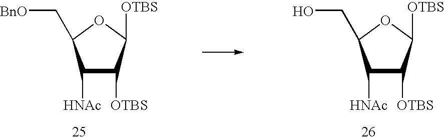

To a solution of 3-N-acetyl-3-deoxy-5-O-benzyl-1,2-O-bis-(tert-butyldimethylsilyl)-β-D-ribofuranose (0.361 g, 0.0708 mmole) in MeOH (3 mL) was added Pd/C (about 25 mg). The mixture was subjected to 50 psi H₂ for 64 h. The reaction mixture was filtered and the filtrate concentrated under reduced pressure to afford the crude product. The crude product was purified via chromatography (silica gel, gradient 1:1→3:1 EtOAc/hexanes→2:1:1 EtOAc/hexanes/MeOH) to afford 3-N-acetyl-1,2-OTBS ribose in 64% yield. The product was analyzed by TLC (silica gel, 1:1 hexanes/EtOAc).

### 3-N-Acetyl-3-deoxy-1,2-O-bis-(tert-butyldimethylsiyl)-β-D-ribofuranose (26)

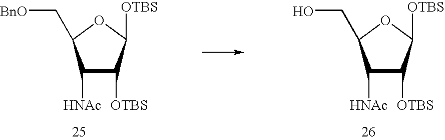

To 3-N-acetyl-3-deoxy-5-O-benzyl-1,2-O-bis-(tert-butyldimethylsilyl)-β-D-ribofuranose (0.278 g, 0.545 mmol) in 22.7 mL EtOH was added 10% Pd/C (0.185 g). The reaction was stirred under 50 psi H₂ for 2 days. The Pd/C was filtered off through Celite and washed with EtOH. The solvent was evaporated and dried in vacuo. Flash chromatography on silica (1:1 Hexanes/EtOAc) afforded the product as a white solid (0.203 g, 89%). ¹H NMR (CDCl₃) δ 6.08 (bd, J=8.7 Hz, 1H), 5.13 (s, 1H), 4.47-4.40 (m, 1H), 3.99 (d, J=4.8 Hz, 1H), 3.93-3.87 (m, 1H), 3.72-3.67 (m, 2H), 3.40 (dd, J=8.9, 5.1 Hz, 1H), 2.02 (s, 3H), 0.95 (s, 9H), 0.89 (s, 9H), 0.14 (s, 6H), 0.11 (s, 3H), 0.10 (s, 3H); ¹³C NMR (CDCl₃) δ 170.8, 102.2, 84.3, 78.6, 64.6, 53.1, 25.8, 25.7, 23.3, 18.2, 17.9, −4.1, −4.4, −4.9, −5.2. HRMS-ESI: calcd for C₁₉H₄₁NO₅Si₂ (M+Na⁺), 442.2421, obsd 442.2437.

### 3-N-Acetyl-3-deoxy-1,2-O-bis-(tert-butyidimethylsilyl)-β-D-ribofuranose 5-hydrogen phosphate (27)

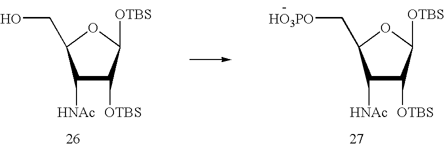

This compound was prepared according to the following literature procedure: J. Am. Chem. Soc. 105 (25) 7428-35 (1983). To a solution of POCl₃ (4.4 μL, 0.0483 mmole) in acetonitrile (16 μL) at 0° C. was added pyridine (20.8 μL, 0.0204 mmole). To this solution was added a solution of 3-N-acetyl-3-deoxy-1,2-O-bis-(tert-butyldimethylsilyl)-β-D-ribofuranose (0.0135 g, 0.0332 mmole) in acetonitrile (16 μL). Additional acetonitrile (16 μL) was used to aid in the transfer. The reaction mixture was stirred at 0° C. for 2 h, then water added (150 μL). After about 45 min, the reaction mixture concentrated under reduced pressure. The residue was dissolved in water and the pH adjusted to about 7 with 1M NaOH, and the solution lyophilized to afford the product.

### 3-N-Acetyl-3-deoxy-1,2-O-bis-(tert-butyidimethylsilyl)-β-D-ribofuranose 5-hydrogen phosphate (TEA salt) (27)

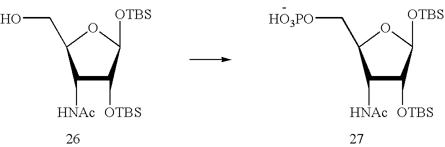

To 3-N-acetyl-3-deoxy-1,2-O-bis-(tert-butyldimethylsilyl)-β-D-ribofuranose (0.100 g, 0.239 mmol) in 650 μL dry THF at 0° C. was added triethylamine (0.217 g, 2.15 mmol), followed by POCl₃ (0.055 g, 0.358 mmol) drop wise. After stirring at 0° C. for 2 h, a few ice chips were added and stirred for an additional hour. The solvent was evaporated and the residue dried in vacuo. The desired product was purified from free inorganic phosphate using reversed-phase flash chromatography and a step-wise gradient of MeOH (25-100% in degassed H₂O) (Kühler and Lindsten, 1983, J. Org. Chem. 48: 3589-3591; O'Neil, 1991, Synlett 9: 661-662). After identifying the desired fractions by the phosphatase assay (described above in general procedures), the fractions were combined to yield 3-N-acetyl-3-deoxy-1,2-O-bis-(tert-butyidimethylsilyl)-β-D-ribofuranose 5-hydrogen phosphate as the TEA salt (0.129 g, 90%). ¹H NMR (CDCl₃) δ 6.73 (bd, 1H), 5.01 (s, 1H), 4.21-4.11 (m, 2H), 4.07 (d, J=3.8 Hz, 1H), 4.02-3.89 (m, 2H), 3.01 (bq, 6H), 1.95 (s, 3H), 1.25 (bt, 9H), 0.83 (s, 9H), 0.82 (s, 9H), 0.03 (s, 6H), 0.00 (s, 6H); ¹³C NMR (CDCl₃) δ 170.5, 103.3, 80.0, 68.32, 68.26, 53.8, 45.5, 25.9, 25.8, 23.4, 18.2, 18.0, 8.6, −4.0, −4.77, −4.82, −5.1; ³¹P NMR (CDCl₃) δ 2.3. HRMS-ESI: calcd for C₁₉H₄₂NO₈PSi₂ (M−H)⁻, 498.2114, obsd 498.2107.

### 3-N-Acetyl-3-deoxy-D-ribofuranose 5-hydrogen phosphate (TEA salt) (28)

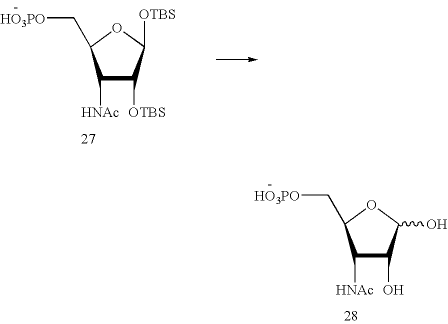

To 3-N-Acetyl-3-deoxy-1,2-O-bis-(tert-butyidimethylsilyl)-β-D-ribofuranose 5-hydrogen phosphate (TEA salt) (0.0229 g, 0.0381 mmol) in 340 μL ACN was added 190 μL Dowex-50WX2-100 (H+ form) (as a 1:1 slurry with H₂O). The reaction was stirred for 2 days. The reaction was passed over Dowex-50WX8 (TEA form) and the solvent was evaporated and the residue dried under vacuum. The desired product was passed over a reversed-phase silica plug and washed with H₂O (5 mL). The solvent was evaporated to yield 3-N-acetyl-3-deoxy-D-ribofuranose 5-hydrogen phosphate as the TEA salt (0.0133 g, 94%). A mixture of α and β anomers (1:3 ratio) were observed by NMR. ¹H NMR [distinct signals for the anomeric position (C-1) could be discerned] (D₂O) δ 5.44 (d, J=3.6 Hz, 0.25H), 5.27 (s, 0.75H), 4.40-4.29 (m, 1H), 4.24-4.19 (m, 1H), 4.16-4.09 (m, 1H), 4.07-3.95 (m, 1H), 3.92-3.84 (m, 1H), 3.18 (q, J=7.4 Hz, 6H), 2.03 (s, 3H), 1.25 (t, J=7.4 Hz, 9H); ¹³C NMR [signals for both anomers could be discerned for the ribofuranose carbons and the acetyl carbonyl only] (D₂O) δ 174.7, 174.5, 101.9, 96.9, 79.7, 79.5, 74.4, 74.3, 69.9, 66.5, 51.8, 51.3, 46.8, 22.0, 8.4; ³¹P NMR (CDCl₃) δ 1.0. HRMS-ESI: calcd for C₇H₁₄NO₈P (M−H)⁻, 270.0384, obsd 270.0417.

Bisphosphonate Synthesis

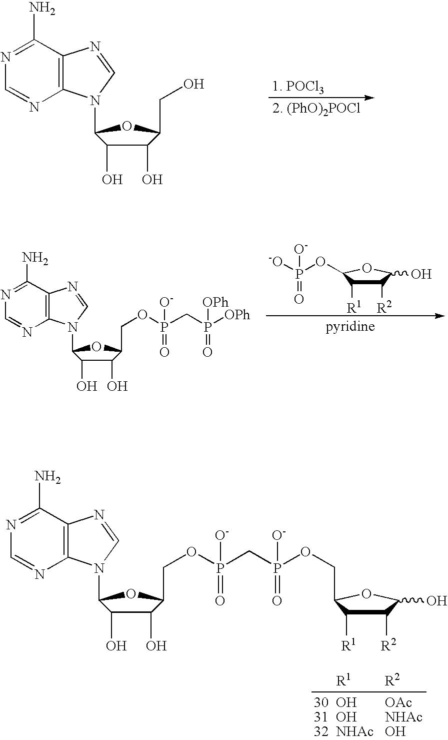

Methylenebisphosphonate Synthesis

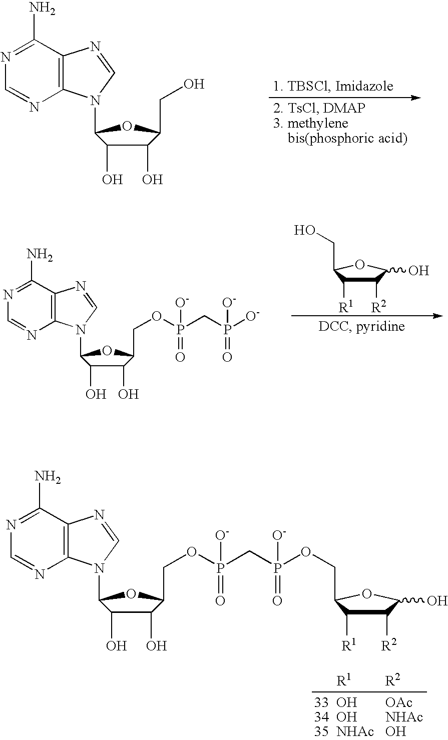

Methodology for the couplings to make the above phosphonate and methylene phosphonates can be found in the following references: Michaelson, 1964, Biochim Biophys Acta 91: 1-13; van der Wenden et al., 1998, J. Med. Chem. 41: 102-108; Davisson et al., 1987, J. Org. Chem. 52: 1794-1801; Pankiewicz et al., 1997, J. Am. Chem. Soc. 119: 3691-3695; and Ma et al., 1989, Bioorg. Chem. 17:194-206.

O-Acetyl-ADP-ribose (2′-OAADPr) (30)

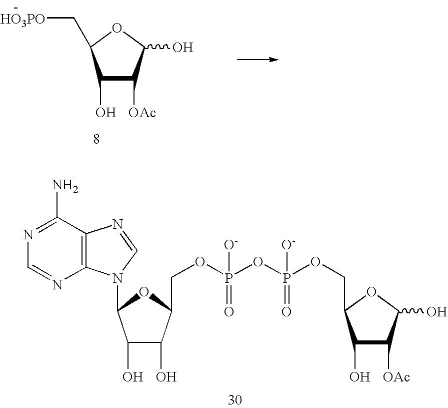

The tri-n-octylammonium salt of AMP (prepared via titration of an equal molar amount of AMP and tri-n-octylamine in MeOH) (Michelson, 1964, Biochim. Biophys. Acta 91: 1-13) (0.0206 g, 0.0294 mmol) was co-evaporated from 100 μL DMF to remove trace water and brought up in a final volume of 150 μL DMF. Diphenylphosphochloridate (0.0118 g, 0.0441 mmol) was added to the reaction, followed immediately by tributylamine (0.0109 g, 0.0587 mmol). After stirring for 3 h, the solvent was evaporated in vacuo. The resulting residue was chilled to 0° C. and 750 μL ether was added with shaking to precipitate the desired product. After 30 min, the ether was removed by decantation and the remaining precipitate was co-evaporated from DMF (100 μL). Upon drying under vacuum, 60 μL DMF was added to the activated AMP and 2-O-Acetyl-D-ribofuranose 5-hydrogen phosphate (TEA salt)) (0.0219 g, 0.0587 mmol) in 150 μL DMF was added, followed immediately by 460 μL pyridine. After stirring for 1 day, the solvent was evaporated off and the crude material was dried under vacuum. The crude material was dissolved in 40 mL 10 mM NH₄OAc and loaded to a column of DEAE-cellulose (Whatman DE52) (2.5×50 cm); after washing with 50 mL of buffer, a linear gradient was formed between 10 and 500 mM NH₄OAc (250 mL each, pH 4.8) and fractions were collected (10 mL). An additional 50 mL 500 mM NH₄OAc was passed over the column. The absorbance was measured at 260 nm and fractions containing the desired product were combined and lyophilized. O-Acetyl-ADP-ribose eluted in the first major band of material (the second band of material contained diadenosine 5′-pyrophosphate (AppA). The resulting material contained contaminants and further purified by preparative HPLC using methodology previously developed (Jackson and Denu, 2002, J. Biol. Chem. 277: 18535-18544).

### 2′-N-Acetyl-ADP-ribose (2′-NAADPr) (31)

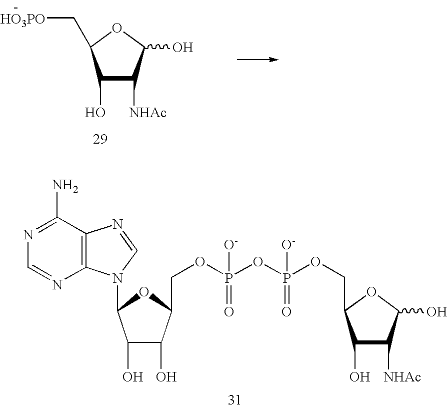

A procedure similar to that described above for O-acetyl-ADP-ribose was carried out containing the tri-n-octylammonium salt of AMP (0.0198 g, 0.0282 mmol) with 2-N-acetyl-2-deoxy-D-ribofuranose 5-hydrogen phosphate (TEA salt) (0.0210 g, 0.0564 mmol). The crude material was dissolved in 40 mL 10 mM NH₄HCO₃ (pH 8) and loaded to a column of DEAE-cellulose (Whatman DE52) (2.5×30 cm); after washing with 50 mL 10 mM NH₄HCO₃, a linear gradient was formed between 10 and 500 mM NH₄HCO₃ (250 mL each) and fractions were collected (10 mL). An additional 50 mL 500 mM NH₄HCO₃ was passed over the column. The absorbance was measured at 260 nm and fractions containing the desired product were combined and lyophilized. 2′-N-Acetyl-ADP-ribose eluted in the first band of material (the second band of material contained diadenosine 5′-pyrophosphate (AppA). The resulting material contained contaminants (including AppA) which were removed by rechromatography with preparative HILIC, followed by preparative C18. The peak corresponding to the desired product was collected and lyophilized after each column to give the desired product (2′-N-acetyl-ADP-ribose) as a white flocculent powder (0.0025 g, 14.8%): Analytical HPLC (HILIC) retention time=23.1 min; analytical HPLC (C18) retention time=15.4 min. A combination of N-acetyl rotomers and α and β anomers (1:1 ratio) was observed by NMR. ¹H NMR [distinct signals for the anomeric position (C-1′) and acetyl could be discerned] (D₂O) δ 8.62 (s, 1H), 8.41 (s, 1H), 6.14 (d, J=5.3 Hz, 1H), 5.45-5.41 (m, 0.5H), 5.25-5.22 (m, 0.5H), 4.74-4.70 (m, 1H), 4.54-4.50 (m, 1H), 4.40-4.37 (m, 1H), 4.34-4.30 (m, 1H), 4.28-4.20 (m, 3H), 4.20-4.15 (m, 1H), 4.12-4.08 (m, 1H), 4.05-4.02 (m, 1H), 2.04, 2.03, 2.00 (s, s, s, 3H); ¹³C NMR [multiple signals were observed for each carbon due to the combination of rotomers/anomers] (D₂O) δ 177.2, 177.0, 176.8, 165.7, 165.4, 165.2, 152.5, 150.8, 147.4, 144.8, 121.0, 120.8, 120.1, 117.8, 115.4, 104.4, 102.7, 99.3, 98.2, 90.5, 86.8, 86.7, 86.44, 86.38, 84.94, 84.87, 84.8, 84.6, 82.03, 81.99, 81.9, 81.8, 77.3, 76.7, 72.8, 72.4, 72.3, 72.0, 70.1, 69.0, 68.8, 68.4, 67.7, 65.1, 60.3, 56.5, 54.4, 54.0, 24.5, 24.44, 24.42, 24.40; ³¹P NMR (D₂O) δ −10.4 (m). HRMS-ESI: calcd for C₁₇H₂₅N₆O₁₄P₂⁻ (M−H)⁻, 599.0909, obsd 599.0875.

### 3′-N-Acetyl-ADP-ribose (3′-NAADPr) (32)

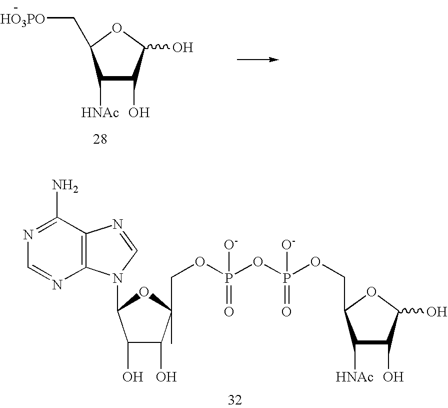

A procedure very similar to that described for O-acetyl-ADP-ribose was carried out containing the tri-n-octylammonium salt of AMP (0.0125 g, 0.0179 mmol) with 3-N-acetyl-3-deoxy-D-ribofuranose 5-hydrogen phosphate (TEA salt) (0.0100 g, 0.0269 mmol). Purification of the desired product was carried out utilizing the same gradients as performed for 2′-N-acetyl-ADP-ribose. The peak corresponding to the desired product after preparative HPLC was collected and lyophilized to give the desired product (3′-N-Acetyl-ADP-ribose) as a white flocculent powder (0.0017 g, 15.9%): Analytical HPLC (HILIC) retention time=23.9 min; analytical HPLC (C18) retention time=15.0 min. A mixture of α and β anomers (1:3 ratio) were observed by NMR. ¹H NMR [distinct signals for the anomeric position (C-1′) could be discerned] (D₂O) δ 8.62 (s, 1H), 8.40 (s, 1H), 6.15 (d, J=5.4 Hz, 1H), 5.41 (d, J=3.6 Hz, 0.25H), 5.25 (s, 0.75H), 4.74 (t, J=5.2 Hz, 1H), 4.53 (t, J=4.4 Hz, 1H), 4.41-4.37 (m, 1H), 4.34-4.30 (m, 1H), 4.27-4.19 (m, 3H), 4.17-4.13 (m, 1H), 4.12-4.06 (m, 1H), 4.03-3.92 (m, 1H), 2.00 (s, 3H); ¹³C NMR [distinct signals for the α and β anomers could be discerned for multiple carbons] (D₂O) δ 177.1, 165.8, 165.5, 152.9, 151.1, 147.9, 144.9, 122.5, 120.2, 117.8, 115.5, 104.4, 99.4, 90.6, 86.8, 82.1, 81.9, 77.3, 76.8, 72.9, 72.4, 70.2, 68.8, 67.7, 54.5, 54.0, 24.6, 24.5; ³¹P NMR (D₂O) δ −10.4 (m). HRMS-ESI: calcd for C₁₇H₂₅N₆O₁₄P₂⁻ (M−H)⁻, 599.0909, obsd 599.0886.

Alternate Methylenebisphosphonate Synthesis

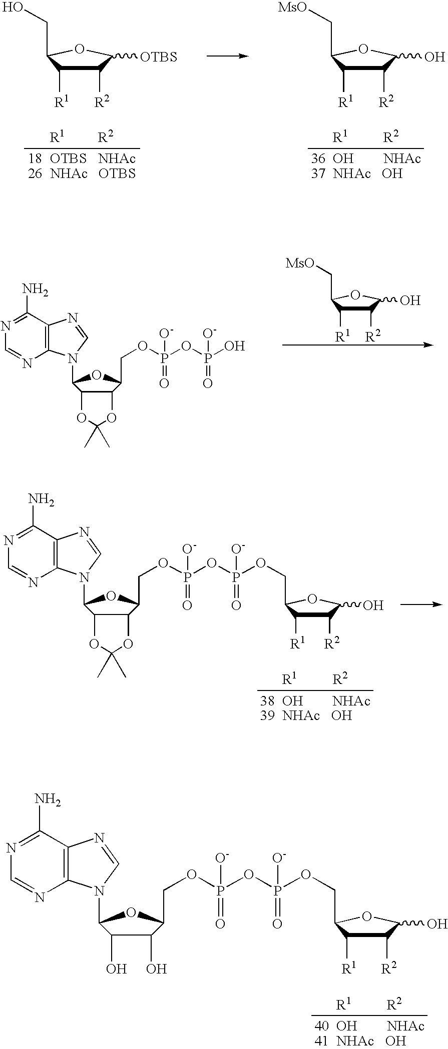

Mesylation of ribose sugars 18 and 26 may be done according to the procedure of Yi et al., 2005, Tetrahedron 61: 11716-11722, followed by deprotection of the t-butyldimethylsilyl groups according to the procedure in Corey et al., 1980, Tetrahedron Lett. 21: 137-140, to afford mesylates 36 and 37.

The 2′,3′-O-isopropylideneadenosine-methylenebis (phosphonate) may be synthesized by the condensation of 2′,3′-O-Isopropylidene-5′-O-toluenesulfonyladenosine with tris(tetra-n-butylammonium)hydrogen methanediphosphonate according to the procedure of Zhou et al., 2004, J. Am. Chem. Soc. 126: 5690-5698 (for complete characterization, see Lesiak et al., 1998, J. Org. Chem. 63: 1906-1909).

The methylenebisphosphonate may be alkylated with a ribose sugar mesylate such as 36 or 37 according to the procedure of Zhou et al., 2004, J. Am. Chem. Soc. 126: 5690-5698 to afford compounds such as 38 or 39. Deprotection of the isopropylidene moiety with Dowex, according to the procedure of Lesiak et al., 1998, J. Org. Chem. 63: 1906-1909, may then afford compounds such as 40 or 41.

HPLC Stability Studies

To validate the installation of an N-acetyl as a non-hydrolyzable substitution, the stabilities of both 2′- and 3′-NAADPr were evaluated at physiological temperature and pH using conditions previously described (Borra et al., 2002, J. Biol. Chem. 277: 12632-12641). Results previously obtained with OAADPr indicated that approximately 18% was hydrolyzed to ADPr within 3 h (Borra et al., 2002, J. Biol. Chem. 277: 12632-12641). HPLC analysis of both 2′- and 3′-NAADPr using exact conditions indicated negligable decomposition (<1%) over 3 days and confirmed the stability of the acetyl funcitonality to spontaneous hydrolysis at physiological pH.

Reactions were carried out with both analogs (final concentration of 500 μM) in 50 mM Tris (pH 7.5 at 37° C.) containing 1 mM DTT. Samples were incubated at 37° C. over 3 days and four time points were collected (0, 24, 48, and 72 h). Each sample was quenched with H₂O containing 0.05% TFA and analyzed by HPLC (analytical C18) using the method described in general procedures. Each trace was integrated (Shimadzu EZStart version 7.2.1 SP1) to determine peak area and analog percentage is shown in the following table 1. Both 2′- and 3′-NAADPr contained trace impurities at 12.9 and 12.5 min respectively and contributed to the observed degradation products visible at 23.3 min in each study.

TABLE 1
NAADPr Stability at Physiological Temperature and pH

| | 0 h | 24 h | 48 h | 72 h |
|---|---|---|---|---|
| 2′-NAADPr | 98.7% | 98.7% | 98.2% | 98.3% |
| 3′-NAADPr | 95.7% | 98.2% | 98.0% | 98.0% |

Channel Gating

Patch clamp electrophysiology is performed as described in Perraud et al., 2001, Nature 411: 595-599. Briefly, cells are patch clamped in the whole cell configuration at 25° C. using pipettes with resistances ranging from 2-3 MOhms. Currents are recorded on an EPC9 patch clamp amplifier with automatic capacitance compensation using a protocol generating a voltage ramp from −100 to +100 mV every two seconds at a holding potential of 0 mV. Bath solutions include 150 mM NaCl, 2.8 mM KCl, 5 mM CsCl, 1 mM CaCl₂, 2 mM MgCl₂, and 10 mM Hepes (pH 7.2). Pipette solutions include 135 mM CsGlutamate, 1 mM MgCl₂, 8 mM NaCl, 10 mM Hepes (pH 7.2), and 10 mM EGTA. For low resolution presentation of current development over the course of the experiment, instantaneous currents at −80 mV are extracted from each ramp and plotted versus time.

Cell Culture

Tetracycline-inducible HEK-293 TRPM2-expressing cells (Perraud et al., 2001, Nature 411: 595-599) are cultured at 37° C. with 5% CO₂ in DMEM supplemented with blasticidin (5 μg ml⁻¹; Invitrogen) and zeocin (0.4 mg/ml; Invitrogen). Wild-type HEK-293 cells are cultured at 37° C. with 5% CO₂ in DMEM. To induce expression of TRPM2, cells are treated with tetracycline 1 μg mL⁻¹ (Invitrogen) for 24 h.

Conversion Assays with OAADPr

Cytoplasmic and nuclear extracts are prepared as described in Rafty et al., 2002, J. Biological Chemistry 277: 47114-47122. For cytoplasmic esterase activity, the standard incubation mixture (50 μl) contains 10 μl of crude HEK-293 cytoplasmic extract 200 μM O—[³H]AADPr, followed by the addition of 0-100 μM puromycin for 30 min. The reaction is quenched by transferring 40 μL aliquots of this reaction to 50 μL of activated charcoal slurry, PBS pH 7.0. Tubes are vortexed and centrifuged. Aliquots (50 μL) of the supernatant are removed and transferred to a new tube. Tubes are centrifuged and 40 μL of the supernatant is removed and analyzed by liquid scintillation counting. The presence of an enzymatic activity in HEK-293 cell nuclei that can metabolize OAADPr is quantitated by incubating O—[³H]AADPr with HEK 293 cell nuclear extract, with and without the compound of the present invention and monitoring the loss of radioactivity in the O—[³H]AADPr peak when resolved by reverse-phase HPLC. The standard incubation mixture (50 μL) contained 10 μL of crude HEK-293 nuclear extract 200 μM 0-[³H]AADPr, followed by the addition of 0 or 100 μM the compound of the present invention for 60 min. After 60 min the reaction is terminated by addition of TFA (final concentration 1%). All samples are injected onto a Beckman Biosys 510 HPLC system and a Vydac C18 (1.0 Å˜25 mm) small pore preparative column (Vydac, Hesperia, Calif.) as previously described (Rafty et al., 2002, Journal of Biological Chemistry 277: 47114-47122). A charcoal binding assay for OAADPr esterase can also be used. The standard incubation mixture (50 μL) contains 10 μL of crude HEK-293 cytoplasmic extract, 200 μM O—[³H]AADPr, followed by the addition of 0-100 μM puromycin for 30 min. The reaction is quenched by transferring 40 μL aliquots of this reaction to 50 μL of activated charcoal slurry, PBS pH 7.0. Tubes are vortexed and centrifuged. Aliquots (50 μL) of the supernatant are removed and transferred to a new tube. Tubes are centrifuged and 40 μL of the supernatant is removed and analyzed by liquid scintillation counting.

Cell Viability Assays

Wild-type or tetracycline induced HEK-293 cells are cultured in 6 well plates at a cell density of 500,000. When cells are 60-70% confluent, cells are re-fed with media containing 1 μg/mL tetracycline. Twenty-four hours post tetracycline induction, cells are treated with various concentrations of the compound of the present invention for 16 hours. Cell monolayers are washed with PBS, and 500 μL of a 2 μM calcein stock solution is added directly to the cells for 10 minutes. Fluorescence in the cell samples is measured using a microplate reader with the excitation and emission filters set at 485 nm and 530 nm respectively.

Liquid Chromatography Tandem Mass Spectrometry Analyses

Cellular levels of OAADPr are determined by LC MS MS (Ion trap with MRM) analysis of cell extracts. Cell monolayers are washed with ice-cold phosphate buffered saline, pH 7.4 and removed from the culture dish by scraping. Cells are pelleted by centrifugation using a clinical centrifuge for 10 min at 4° C. The cell pellet is lysed in 10% trifluoroacetic acid in water. Cellular debris is removed by centrifugation, and the supernatant is frozen and lyophilized to dryness. The residue is resuspended in 50% acetonitrile/water and analyze by LC-MSMS using hydrophilic interaction chromatography on the front end of an ion trap.

MacroH2A1.1 Binding

MacroH2A1.1 is an atypical histone variant associated with heterochromatin and implicated in transcriptional repression. Recent calorimetry evidence indicates that a 1:1 mixture of the 2′- and 3′-OMDPr is capable of binding to the human macroH2A1.1 domain with a K_{d} of approximately 2 μM and an enthalpy of −17.8 kcal/mol (Kustatcher et al., 2005, Nat. Struct. Mol. Biol. 12: 624-625). Using a method similar to that described by Kustatcher, the binding of ADPr to MacroH2A1.1 was determined to have a K_{d} of 2.4 μM and an enthalpy of −15.4 kcal/mol. The splice-variant MacroH2A1.2 was also studied, but no binding of ADPr was observed. The binding of 2′- and 3′-NAADPr may be evaluated using a protocol similar to that described by Kustatcher.

Nudix Hydrolases

The Nudix hydrolases, mNudT5 and YSA1, and NudT9 are a family of ADPr metabolizing enzymes. It has been shown that mNudT5 and YSA1 efficiently hydrolyze OAADPr in vitro to AMP and acetylated ribose 5′-phosphate, whereas NudT9 was 500-fold less efficient as compared to ADPr (Rafty et al., 2002, J. Biol. Chem. 277: 47114-47122). Reaction mixtures containing ADPr (5-300 μM) were incubated with NudT9 (100 ng) or YSA1 (10 ng). Michaelis-Menten plots were generated for the hydrolysis of ADPr by NudT9 and YSA1. For NudtT9, k_{cat} was determined to be 9.3 s⁻¹ and Kₘ was 23.9 μM. For YSA1, k_{cat} was determined to be 6.5 s⁻¹ and Kₘ was 39.9 μM. The hydrolysis of 2′- and 3′-NAADPr is evaluated using a protocol similar to that described by Rafty.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters normally encountered in biochemistry, and obvious to those skilled in the art, are within the scope of this invention. All publications, patents, and patent applications cited herein are incorporated by reference in their entirety for all purposes.

We claim:

1. A compound of the formula (1), or a salt thereof: 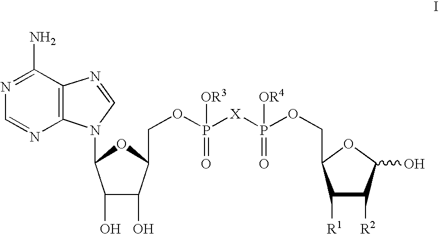
where
X is O or CH₂; and
R¹ and R² are each independently selected from the group consisting of hydrogen, —F, —OH, —OR^{a}, —SR^{a}, —NH₂, —NHC(O)CH₃, —CH₂C(O)CH₃ and —CH₂C(CH₃)═CH₂;
where R^{a} is selected from the group consisting of substituted or unsubstituted C₁₋₈ alkyl, substituted or unsubstituted C₂₋₈ alkenyl, substituted or unsubstituted C₂₋₈ alkynyl; and
R³ and R⁴ are each independently selected from the group consisting of hydrogen, substituted or unsubstituted C₁₋₈alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl; with the proviso that at least one of R¹ and R² is other than hydrogen, —F, —OH, or —NH₂;
with the proviso that at least one of R¹ and R² is other than hydrogen, —F, —OH, or —NH₂; and
with the proviso that excluded from the scope of formula I is O-acetyl ADP ribose.

2. The compound of claim 1, where R¹ is selected from the group consisting of —NHC(O)CH₃, —CH₂C(O)CH₃ and —CH₂C(CH₃)═CH₂.

3. The compound of claim 1, where R¹ is —NHC(O)CH₃.

4. The compound of claim 1, where R² is selected from the group consisting of —NHC(O)CH₃, —CH₂C(O)CH₃ and —CH₂C(CH₃)═CH₂.

5. The compound of claim 1, where R² is —NHC(O)CH₃.

6. The compound of claim 1, where X is CH₂.

7. The compound of claim 1, which is represented by formula (II) or a salt thereof:

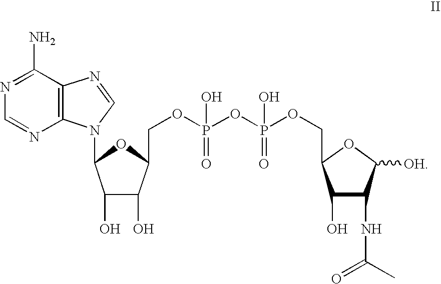

8. The compound of claim 1, which is represented by formula (III) or a salt thereof:

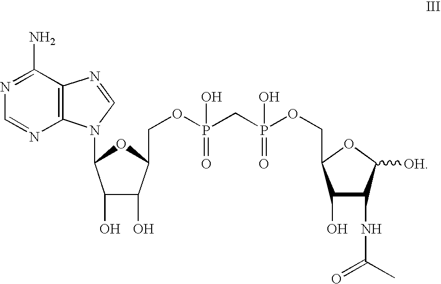

9. The compound of claim 1, which is represented by formula (IV) or a salt thereof:

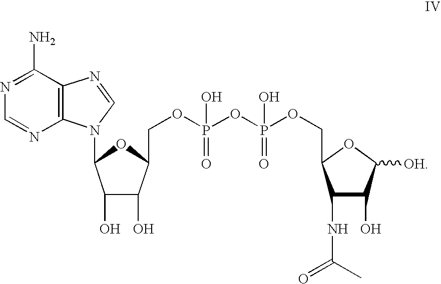

10. The compound of claim 1, which is represented by formula (V) or a salt thereof:

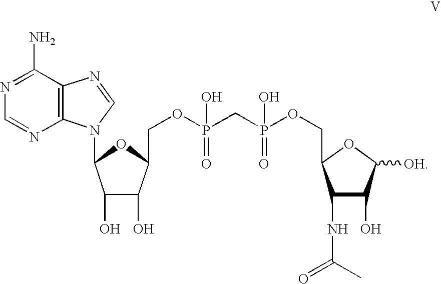

11. A composition comprising a pharmaceutically acceptable carrier and a compound according to claim 1.

12. A composition comprising a pharmaceutically acceptable carrier and a compound according to claim 8.

13. A composition comprising a pharmaceutically acceptable carrier and a compound according to claim 10.

14. A method for modulating cell death in a target cell comprising:
contacting a cell expressing O-acetyl-ADP-ribose with a compound of formula (I), or a salt thereof: 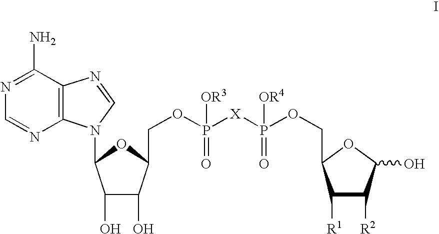
where
X is O or CH₂; and
R¹ and R² are each independently selected from the group consisting of hydrogen, —F, —OH, —OR^{a}, —SR^{a}, —NH₂, —NHC(O)CH₃, —CH₂C(O)CH₃ and —CH₂C(CH₃)═CH₂;
where Ra is selected from the group consisting of substituted or unsubstituted C₁₋₈ alkyl, substituted or unsubstituted C₂₋₈ alkenyl, substituted or unsubstituted C₂₋₈ alkynyl; and
R³ and R⁴ are each independently selected from the group consisting of hydrogen, substituted or unsubstituted C₁₋₈alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
with the proviso that at least one of R¹ and R² is other than hydrogen, —F, —OH, or —NH₂;
thereby inducing cell death in said target cell
with the proviso that excluded from the scope of formula I is O-acetyl ADP ribose.

15. The method of claim 14, wherein the compound is represented by formula (III) or a salt thereof:

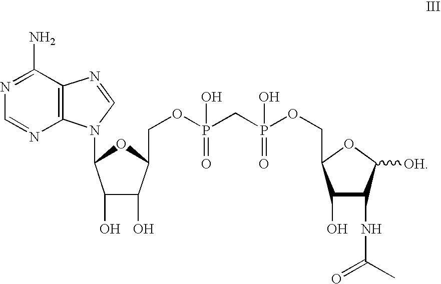

16. The method of claim 14, wherein the compound is represented by formula (V) or a salt thereof:

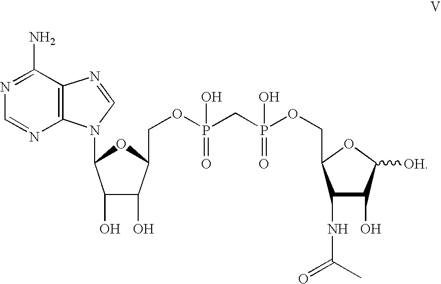

17. A method for treating an O-acetyl-ADP-ribose-mediated condition comprising administering to a subject an effective amount of a compound of formula (I), or a salt thereof: 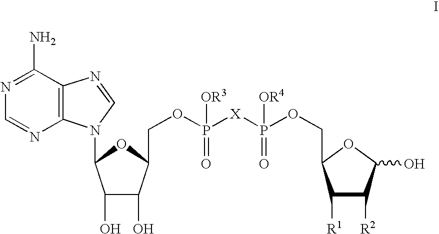
where
X is O or CH₂; and
R¹ and R² are each independently selected from the group consisting of hydrogen, —F, —OH, —OR^{a}, —SR^{a}, —NH₂, —NHC(O)CH₃, —CH₂C(O)CH₃ and —CH₂C(CH₃)═CH₂;
where R^{a} is selected from the group consisting of substituted or unsubstituted C₁₋₈ alkyl, substituted or unsubstituted C₂₋₈ alkenyl, substituted or unsubstituted C₂₋₈ alkynyl; and
R³ and R⁴ are each independently selected from the group consisting of hydrogen, substituted or unsubstituted C₁₋₈alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
with the proviso that at least one of R¹ and R² is other than hydrogen, —F, —OH, or —NH₂;
with the proviso that excluded from the scope of formula I is O-acetyl ADP ribose.

18. The method of claim 17, wherein the compound is represented by formula (III) or a salt thereof:

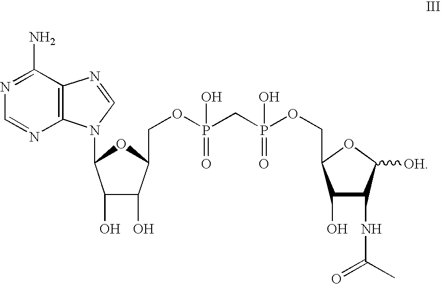

19. The method of claim 17, wherein the compound is represented by formula (V) or a salt thereof:

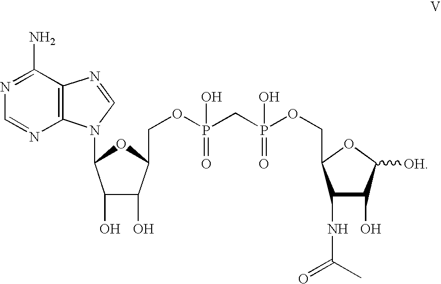

20. The method of claim 17, wherein the O-acetyl-ADP-ribose-mediated condition is cancer.

21. A method for producing 2-N-acetyl-2-deoxy-D-ribofuranose 5-hydrogen phosphate comprising:
providing 1,2:5,6-di-O-isopropylidene-3-O-triflate-α-D-glucofuranose; transforming 1,2:5,6-di-O-isopropylidene-3-O-triflate-α-D-glucofuranose to 2-N-acetyl-2-deoxy-D-ribofuranose 5-hydrogen phosphate; isolating 2-N-acetyl-2-deoxy-D-ribofuranose 5-hydrogen phosphate.

22. The method of claim 21, further comprising transforming 1,2:5,6-di-O-isopropylidene-α-D-glucofuranose to 1,2:5,6-di-O-isopropylidene-3-O-triflate-α-D-glucofuranose.

23. A method for producing 3-N-acetyl-3-deoxy-D-ribofuranose 5-hydrogen phosphate comprising:
providing 5-O-benzyl-3-triflyl-1,2-O-isopropylidene-α-D-xylofuranose; transforming 5-O-benzyl-3-triflyl-1,2-O-isopropylidene-α-D-xylofuranose to 3-N-Acetyl-3-deoxy-D-ribofuranose 5-hydrogen phosphate; and isolating 3-N-Acetyl-3-deoxy-D-ribofuranose 5-hydrogen phosphate.

24. The method of claim 23, further comprising transforming 1,2-O-isopropylidne-α-D-xylofuranose to 5-O-benzyl-3-triflyl-1,2-O-isopropylidene-α-D-xylofuranose.
